(19) United States
(12) Patent Application Publication
(10) Pub. No.: US 2026/0179782 A1
(43) Pub. Date: Jun. 25, 2026

(54) SYSTEMS AND METHODS FOR IMPROVED PREDICTION CALCULATIONS FOR CARDIAC DRUG DISCOVERY

(71) Applicant: UNIVERSITY OF VIRGINIA PATENT FOUNDATION, Charlottesville, VA (US)

(72) Inventors: Taylor Grant Eggertsen, Ann Arbor, MI (US); Jeffrey John Saucerman, Crozet, VA (US)

(21) Appl. No.: 19/359,298

(22) Filed: Oct. 15, 2025

Related U.S. Application Data
(60) Provisional application No. 63/737,032, filed on Dec. 20, 2024.

(51) Int. Cl. G16H 50/50; G16H 70/40

(52) U.S. Cl. CPC G16H 50/50; G16H 70/40

(57) ABSTRACT

A method for predicting drug pathways that modulate cellular phenotypes is provided. The method includes obtaining a logic-based differential equation model of a signaling network relating to a cellular phenotype, comprising nodes representing proteins or mRNAs and edges representing interactions. The method further includes identifying candidate compounds affecting the cellular phenotype, determining protein targets using a drug-target database, and mapping pathways from protein targets to network nodes by searching a protein interaction database and computing ranked pathways by minimizing a cost function based on edge weights. The method includes expanding the model by incorporating mapped pathways, simulating expanded model variants to predict compound effects on the cellular phenotype, and outputting predictions of how candidate compounds modulate the cellular phenotype through identified pathways.

### CROSS-REFERENCE TO RELATED APPLICATIONS

This application claims the benefit of U.S. Provisional Patent Application Ser. No. 63/737,032, filed on Dec. 20, 2024, and entitled “SYSTEMS AND METHODS FOR IMPROVED PREDICTION CALCULATIONS FOR CARDIAC DRUG DISCOVERY,” which is herein incorporated by reference in its entirety.

### STATEMENT OF FEDERALLY SPONSORED RESEARCH

This invention was made with government support under HL162925 awarded by the National Institutes of Health. The government has certain rights in the invention.

### BACKGROUND

Cardiac hypertrophy represents a pathological enlargement of heart muscle cells that serves as a clinical predictor of heart failure. This condition arises in response to various physiological stressors including pressure overload, drug toxicity, and genetic mutations. While several medications prescribed for heart failure can affect cardiac hypertrophy, such as angiotensin-converting enzyme inhibitors, beta blockers, and angiotensin receptor blockers, heart failure rates continue to rise with mortality rates exceeding 40% within five years.

Drug repurposing has emerged as an approach to accelerate therapeutic development and reduce costs by identifying new applications for existing medications. However, experimental approaches to prioritize drugs from large screens and investigate their mechanisms of action are time-intensive and costly. The complexity of hypertrophic signaling, characterized by extensive cross-talk between multiple molecular pathways, further complicates drug development efforts. While protein interaction databases and gene regulatory networks have been used to explore drug-target interactions, these studies have primarily examined network topology rather than simulating network responses to drug treatments. There exists a need for computational methods that can predict drug mechanisms of action and provide mechanistic understanding of how drugs modulate cellular phenotypes.

### SUMMARY OF THE DISCLOSURE

According to an aspect of the present disclosure, a computer-implemented method for predicting drug pathways that modulate cellular phenotypes is provided. The method includes obtaining a logic-based differential equation model of a signaling network relating to a cellular phenotype, the model including a plurality of nodes representing proteins or mRNAs and a plurality of edges representing interactions between the nodes. The method includes identifying a set of candidate compounds that affect the cellular phenotype. The method includes determining protein targets for each candidate compound using a drug-target database. The method includes mapping pathways from the protein targets to nodes in the signaling network by searching a directed protein interaction database and computing a ranked list of pathways by minimizing a cost function based on edge weights along each pathway. The method includes expanding the logic-based differential equation model by incorporating the mapped pathways to create expanded model variants. The method includes simulating each expanded model variant to predict effects of the candidate compounds on the cellular phenotype. The method includes outputting predictions of how the candidate compounds modulate the cellular phenotype through the identified pathways.

According to another aspect of the present disclosure, a drug discovery system is provided. The system includes a processor. The system includes a memory coupled to the processor and storing instructions that, when executed by the processor, cause the processor to access a logic-based model of a signaling network for a given condition, the model including a plurality of nodes and a plurality of edges. The instructions cause the processor to determine a set of candidate compounds that may affect the given condition. The instructions cause the processor to identify protein targets for the candidate compounds using a drug-target database. The instructions cause the processor to determine potential connections from the candidate compounds to the given condition via the logic-based model by computing a ranked list of pathways through searching a directed protein interaction network and minimizing a cost function of edge weights along each pathway. The instructions cause the processor to simulate drug mechanisms of action using logic-based equations accounting for drug action and drug binding properties. The instructions cause the processor to output the ranked list of pathways and predicted drug effects for drug discovery applications.

According to another aspect of the present disclosure, a method of treating cardiomyocyte hypertrophy in a patient is provided. The method includes identifying a drug predicted to inhibit cardiomyocyte hypertrophy using a logic-based machine learning method that maps drug targets to a validated cardiomyocyte hypertrophy signaling network model and predicts antihypertrophic pathways through mechanistic subnetwork analysis. The method includes administering to the patient a therapeutically effective amount of the identified drug, where the drug is selected from the group consisting of escitalopram, mifepristone, tirbanibulin, OSI-930, crizotinib, rifabutin, and hesperidin.

### BRIEF DESCRIPTION OF THE DRAWINGS

FIG. 1 is a flowchart illustrating steps of an example method for predicting drug pathways using a logic-based mechanistic machine learning model.

FIGS. 2A-2D is a schematic diagram showing the LogiRx workflow for identifying pathways from antihypertrophic drugs to a cardiomyocyte hypertrophy signaling network, including drug target identification, pathway mapping, network expansion, and validation processes. (A) Overview of the LogiRx workflow, from drugs to mechanistic predictions. (B) Drug targets are identified from DrugBank and filtered by annotations in the protein-interaction database OmniPath. (C) Drugs and their protein targets are mapped to the hypertrophy network through LogiRx. (D) Drug pathways are individually simulated using Netflux and validated against prior experimental literature. Drug pathways that maintain model validation accuracy are used to expand the hypertrophy network for drug predictions.

FIGS. 3A-3C is a composite figure showing LogiRx-based network model expansion and predictions of how drugs affect cardiomyocyte hypertrophy, including validation results, efficacy predictions across biochemical environments, and regional network modulation patterns. (A) Hypertrophy network variants expanded by individual drug pathways were simulated for their effect on five hypertrophic outputs, as well as validated against 450 experiments from prior literature. (B) Eight drugs were predicted to affect cardiomyocyte hypertrophy with varying efficacy across 17 biochemical environments. (C) Drugs were predicted to inhibit hypertrophy via distinct regional modulation of the hypertrophy signaling network, indicating distinct inhibitory mechanisms.

FIGS. 4A-4E is a series of experimental validation results showing the effects of escitalopram and mifepristone on cardiomyocyte hypertrophy induced by phenylephrine and TGFβ, including representative microscopy images, quantitative cell area measurements, and protein analysis. The effect of five drugs on PE-induced hypertrophy of neonatal rat cardiomyocytes is shown in (A) representative images or (B) quantification of automated segmentation. Escitalopram (20 μM), mifepristone (6 μM), and tirbanibulin (6 μM) significantly prevented PE-induced cell area increase over a 48-h period following treatment. The effect of these five drugs on TGFβ-induced hypertrophy is shown as (C) representative images or (D) quantification of cardiomyocyte cell area by automated segmentation. Escitalopram (20 μM) and mifepristone (6 μM) significantly prevented TGFβ-induced increase in cell area. (E) Western blot of total protein and phospho-PKD in response to PE and/or escitalopram. Two cell isolations were used for each experiment, and a two-way ANOVA was performed followed by a Dunnett's post hoc test. The error bar indicates SEM. * P<0.05, ** P<0.01, *** P<0.001.

FIGS. 5A-5F is a mechanistic analysis figure demonstrating how escitalopram prevents cardiomyocyte hypertrophy by inhibiting serotonin receptors, including subnetwork analysis, simulation results, and experimental validation using serotonin receptor and PI3Kγ inhibitors. (A) Mechanistic subnetwork analysis to predict how escitalopram suppresses cardiomyocyte hypertrophy. (B) Simulations of PE-induced hypertrophy and modulation by escitalopram, Gβγ inhibition, or PI3Kγ inhibition. Serotonin receptor inhibitor sarpogrelate hydrochloride (5 μM) significantly prevents (C) PE- and (D) TGFβ-induced cardiomyocyte hypertrophy 48 h posttreatment, as shown by representative images and quantification of automated segmentation. PI3Kγ inhibitor eganelisib (20 μM) significantly prevents (E) PE- and (F) TGFβ-induced cardiomyocyte hypertrophy 48 h posttreatment, as shown by representative images and quantification of automated segmentation. Two cell isolations were used for each experiment, and a two-way ANOVA was performed followed by a Dunnett's post hoc test. The error bar indicates SEM. * P<0.05, ** P<0.01, *** P<0.001.

FIGS. 6A-6J is an in vivo validation study showing escitalopram's effects on cardiomyocyte hypertrophy in an angiotensin II/phenylephrine mouse model, including treatment protocol, cardiac measurements, histological analysis, and echocardiographic assessments. (A) Schematic of cardiac injury model and escitalopram administration. Mice were continuously delivered AngII/PE through an osmotic minipump to induce cardiac injury and fibrosis and injected with 10 mg/kg/d escitalopram or vehicle control for 14 d. Quantification of HW:BW (B) and LV:BW (C) ratios. (D) Representative picrosirius red staining of heart cross-sectional tissues in sham mice and AngII/PE mice treated with vehicle or escitalopram, and quantification of percent collagen content (E). Results of echocardiographic analyses of AngII/PE mice, showing the left ventricular wall thickness in diastole (F), percent ejection fraction derived from strain analysis (G and H), and the ratio of early mitral valve flow velocity to mitral annulus velocity by tissue Doppler (E/e′) indicating diastolic function. (I) Wheat germ agglutinin staining of tissue sections, with quantification of cardiomyocyte cross-sectional area (J). Statistical analyses performed by one-way ANOVA with Tukey's post hoc multiple comparisons test.

FIGS. 7A-7E is a patient database analysis comparing cardiac hypertrophy incidence in depression patients prescribed escitalopram versus other SSRIs with sex-stratified analyses and echocardiogram verification. (A) Reports of left ventricular hypertrophy (Left) or ventricular hypertrophy (Right) per 1,000 patients are shown for patients with depression taking either escitalopram or another SSRI (fluoxetine, paroxetine, sertraline, or venlafaxine) in the FDA Adverse Events Reporting System. (B) Diagnoses of left ventricular hypertrophy per 1,000 patients are shown for patients with depression taking either escitalopram or another SSRI in the U. Virginia Health System. (C) Verification of echocardiograms from patients in the University of Virginia (UVA) Health System with hypertrophy (prescribed paroxetine) or without hypertrophy (prescribed escitalopram, fluoxetine, sertraline, or venlafaxine). (D) FDA adverse event reports of left ventricular hypertrophy (Left) or ventricular hypertrophy (Right) per 1,000 patients are further divided into incidence by sex. (E) Diagnoses of left ventricular hypertrophy in the UVA Health System, divided into incidence by sex. In the UVA Health System, no female patients prescribed paroxetine and no male patients prescribed buspirone exhibited left ventricular hypertrophy. * P<0.05, ** P<0.01, *** P<0.001.

FIG. 8 is a block diagram of an example system for logic-based machine learning prediction of drug pathways.

FIG. 9 is a block diagram of example components that can implement the system of FIG. 8.

### DETAILED DESCRIPTION

Described here are systems and methods for logic-based mechanistic machine learning prediction of drug pathways that modulate cellular phenotypes. The disclosed systems and methods integrate drug-target databases with protein interaction networks and logic-based differential equation models to predict mechanistic pathways by which drugs affect specific cell states. In some implementations, the disclosed systems may be referred to as a LogiRx system.

In some aspects, the systems and methods may identify novel therapeutic applications for existing drugs by mapping drug targets to validated signaling network models and simulating network responses to drug perturbations. The approach may combine pathway optimization algorithms with mechanistic subnetwork analysis to provide both predictive capabilities and mechanistic understanding of drug action. In some cases, the systems and methods may enable drug repurposing by revealing off-target pathways through which drugs modulate cellular processes, such as cardiac hypertrophy, that differ from their primary therapeutic mechanisms. The predicted drug pathways may be validated through experimental testing in cellular models, animal studies, and analysis of patient databases, providing a comprehensive framework for mechanistic drug discovery and repurposing applications.

As one non-limiting example, in some aspects, the present disclosure can provide systems and methods that include and/or utilize a logic-based predictor of drug pathways, which is configured, trained, and/or programmed to identify how various drugs may impact cardiac conditions or functions, such as inhibiting cardiomyocyte hypertrophy.

In another respect, the present disclosure can provide for a drug discovery system that includes a first memory storage, a second memory storage, a processor, and a memory. The first memory storage may include a first database and the a second memory storage may include a second database. The memory may have stored thereon a set of instructions which, when executed by the processor, cause the processor to obtain a logic-based model of a signaling network relating to a given condition, including a plurality of nodes and a plurality of edges. The processor is further configured to determine a set of candidate compounds that may affect the given condition and identify protein targets for these candidate compounds. Subsequently, the processor determines potential connections from the candidate compounds to the given condition via the logic-based model by computing a rank list of pathways through searching a directed protein interaction network and minimizing a cost function of the product of edge weights along each pathway. Finally, the processor outputs the ranked list to a user for further analysis and drug discovery applications.

In some embodiments, the set of instruction may also cause the processor to use a mechanistic subnetwork to explain drug response and support validation of use of the drug for a given patient or patient population. In some respects, the given condition may be cardiac hypertrophy, whereas in other embodiments other conditions may also be analyzed.

In some cases, the first database may be a drug-target database, such as DrugBank, and the step of determining protein targets uses such database. The second database may be a protein interaction database, such as the Omnipath database, and the step of determining potential connections uses such database.

In another respect, methods of treatment are contemplated herein. For example, a patient having a given condition or at risk of the given condition may be administered a known, approved drug that was identified as affecting the given condition through use of a system as described above. For example, methods of treating human patients suffering from cardiomyocyte hypertrophy or at risk of cardiomyocyte hypertrophy may be administered quantities of known, safe drugs identified through the LogiRx system, such as OSI-930, crizotinib, escitalopram, tirbanibulin, rifabutin, mifepristone, and hesperidin.

In further aspects, the present disclosure contemplates individual pathway models derived from the foregoing systems, and as further described by way of example in the below description of experiments and validation studies.

Thus, the present disclosure contemplates a variety of perspectives on systems and methods for generating models (like LogiRx) that can predict effect of candidate compounds on a given condition; systems and methods for treating patients through use of compounds identified through such models; and systems and methods for supplementing drug discovery, pre-clinical, clinical, and regulatory processes by demonstrating how candidate compounds affect pathways resulting in impact on a given condition.

Logic-based mechanistic machine learning approaches for drug pathway prediction may provide a framework for understanding how pharmaceutical compounds modulate cellular phenotypes through complex signaling networks. Traditional drug discovery methods focus primarily on direct target interactions, but many therapeutic effects occur through indirect pathways that involve multiple protein interactions and regulatory cascades. The disclosed systems and methods address this limitation by integrating multiple data sources to predict and validate mechanistic pathways that connect drug targets to cellular outcomes.

The disclosed approach may combine drug-target databases, protein interaction networks, and logic-based differential equation models to create a comprehensive framework for pathway prediction. Drug-target databases may provide information about known molecular targets for pharmaceutical compounds, while protein interaction databases may contain information about how proteins interact within cellular signaling networks. Logic-based differential equation models may simulate the dynamic behavior of signaling networks and predict how perturbations at specific nodes affect downstream cellular processes. The logic-based modeling may enable simulation of network responses to drug perturbations. These models may incorporate Boolean logic gates and differential equations to represent how signals propagate through cellular networks. When a drug target is perturbed, the model may predict how this perturbation cascades through the network to affect downstream processes such as gene expression, protein activity, and cellular phenotypes.

In some cases, the systems and methods may utilize pathway optimization algorithms to identify the most probable routes by which drugs influence cellular phenotypes. These algorithms may search through directed protein interaction networks to find pathways that connect drug targets to nodes within validated signaling network models. The pathways may be ranked based on cost functions that consider factors such as pathway length, interaction confidence, and network topology.

Mechanistic subnetwork analysis may provide additional insights into drug action by identifying the specific network components that mediate drug effects. This analysis may involve simulating the global response of the network to drug treatment and then systematically testing the contribution of individual network nodes to the observed response. The intersection of drug-responsive nodes and nodes that regulate the drug response may define mechanistic subnetworks that explain how drugs achieve their effects.

The disclosed approach may enable drug repurposing by revealing off-target pathways through which existing drugs modulate cellular processes. Many approved drugs may have effects on cellular pathways that differ from their primary therapeutic mechanisms. By mapping these alternative pathways, the systems and methods may identify new therapeutic applications for existing compounds, potentially accelerating the development of treatments for various conditions.

Validation of predicted drug pathways may occur through multiple approaches, including experimental testing in cellular models, animal studies, and analysis of patient databases. Cellular models may allow for controlled testing of drug effects on specific phenotypes, while animal studies may provide information about drug efficacy in more complex biological systems. Patient database analysis may reveal correlations between drug use and clinical outcomes that support predicted therapeutic effects.

The integration of computational prediction with experimental validation may provide a comprehensive framework for mechanistic drug discovery. This approach may reduce the time and cost associated with traditional drug development by providing mechanistic insights that guide experimental design and prioritize compounds for further testing. The systems and methods may also support regulatory processes by providing mechanistic explanations for observed drug effects.

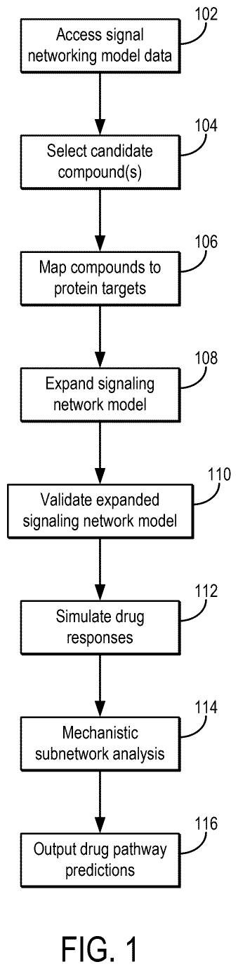

Referring now to FIG. 1, a flowchart is illustrated as setting forth the steps of an example method for predicting drug pathways using a logic-based mechanistic machine learning model. As described above, the machine learning model may provide a comprehensive computational framework for predicting how drugs modulate cellular phenotypes through mechanistic pathway analysis. The method may integrate multiple databases and computational approaches to generate actionable predictions for drug discovery and repurposing applications.

As indicated at step 102, the method may begin by accessing signaling network model data with a computer system. Accessing the signaling network model data may include retrieving such data from a memory or other suitable data storage device or medium, including local databases, cloud-based storage systems, or distributed computing networks. Additionally or alternatively, accessing the signaling network model data may include acquiring such data through real-time database queries, API calls to external repositories, file transfers from remote servers, or direct input from experimental screening results and transferring or otherwise communicating the data to the computer system. The signaling network model data may include logic-based differential equation models, protein interaction networks, drug-target data, protein interaction data, experimental validation datasets, or combinations thereof. In some instances, the signaling network model data may include a pre-existing logic-based signaling network model for a target cellular phenotype.

As noted above, the signaling network model data may include a pre-existing logic-based signaling network model relating to a given condition. The logic-based model may include a plurality of nodes and a plurality of edges that represent the complex interactions within cellular signaling pathways. In some cases, the logic-based model may be constructed based on literature-curated information or other experimental data about protein interactions and regulatory relationships.

In a non-limiting example, the logic-based network model may include nodes representing proteins or mRNAs and edges representing interactions between these molecular components. Each node in the network may correspond to a specific protein or mRNA molecule that participates in the signaling pathway, while each edge may represent a regulatory interaction, such as phosphorylation, binding, or transcriptional regulation, between two nodes. The network structure may capture the hierarchical organization of signaling cascades and the cross-talk between different pathway branches. In some cases, the logic-based network model may incorporate Boolean logic gates and mathematical equations (e.g., differential equations) to simulate the dynamic behavior of the signaling network. Each node in the network may be governed by logic-based equations that determine how the activity or expression level of that node responds to inputs from upstream regulators. These equations may account for different types of regulatory interactions, including activation, inhibition, and cooperative binding effects.

The logic-based network model may serve as a foundation for pathway mapping and drug mechanism prediction. When combined with drug-target databases and protein interaction networks, the model may enable the identification of pathways through which drugs influence cellular processes. The model structure may allow for simulation of drug perturbations at specific nodes and prediction of how these perturbations propagate through the network to affect downstream cellular outcomes.

One or more candidate compounds to be analyzed is then selected at step 104, by receiving or otherwise accessing relevant data including a set of candidate compounds to be analyzed. The selection of candidate compounds may be based on various criteria, including compounds identified from previous experimental screens, compounds with known therapeutic effects, or compounds selected from chemical libraries or databases. In some embodiments, the candidate compounds may be selected from a previous high-throughput screen for compounds that inhibit a specific cellular phenotype, such as cardiomyocyte hypertrophy. For example, the candidate compounds may include those that have demonstrated efficacy in reducing cell area, protein expression markers, or other phenotypic indicators while maintaining acceptable safety profiles. The system may filter candidate compounds based on predetermined criteria such as binding affinity, clinical relevance, regulatory approval status, or known safety profiles. Additionally, the selection process may prioritize compounds that are already approved for human use in other therapeutic areas, thereby facilitating potential drug repurposing applications. The candidate compounds may be stored in a database or data structure that includes associated metadata such as chemical structure, molecular weight, known targets, therapeutic indications, and pharmacokinetic properties.

The method may then proceed to step 106 by mapping the candidate compounds to protein targets using curated drug-target data in the signaling network model data. In some aspects, this step may involve identifying all known protein targets for a given set of candidate compounds, including both primary therapeutic targets and secondary off-target interactions. As a non-limiting example, the mapping process may utilize comprehensive drug-target databases such as DrugBank, ChEMBL, or other curated repositories that contain experimentally validated binding data, pharmacological annotations, and molecular interaction profiles. The system may filter these drug-target relationships based on multiple criteria including binding affinity data, dissociation constants, clinical relevance, therapeutic concentration ranges, or other pharmacokinetic and pharmacodynamic parameters to focus on the most relevant molecular interactions. Additionally, the mapping process may incorporate confidence scores or reliability metrics associated with each drug-target interaction, allowing the system to prioritize high-confidence relationships while maintaining awareness of potential interactions with lower certainty. The system may also consider tissue-specific expression patterns of target proteins, subcellular localization data, and pathway context to ensure that identified drug-target relationships are biologically relevant to the cellular phenotype under investigation. In some embodiments, the mapping step may include cross-referencing multiple databases to validate drug-target interactions and resolve discrepancies between different data sources, thereby improving the accuracy and completeness of the drug-target mapping process.

At step 108, the method may expand the pre-existing signaling network model to incorporate the identified drug targets. This expansion process may utilize protein interaction data contained in the signaling network model data to identify pathways connecting drug targets to nodes within the established signaling network. The expansion may involve systematically searching through comprehensive protein interaction databases to discover both direct and indirect connections between drug targets and network components. In some cases, pathway optimization algorithms such as PathLinker may be employed to rank potential connection pathways based on edge weights and pathway scores, ensuring that the most biologically relevant connections are prioritized for network integration. The PathLinker algorithm may implement graph-based optimization techniques to identify top-scoring directed pathways by minimizing cost functions that account for the cumulative weights of edges along each potential pathway. The system may generate multiple expanded network variants by incorporating different combinations of high-scoring pathways, allowing for comprehensive evaluation of alternative network architectures. Each expanded network variant may be assigned confidence scores based on the reliability of the underlying protein interaction data, pathway scoring metrics, and consistency with known biological mechanisms. The expansion process may also consider pathway length constraints, ensuring that connections between drug targets and network nodes remain within biologically plausible distances while maintaining mechanistic relevance. Additionally, the system may filter potential pathways based on tissue-specific expression patterns, subcellular localization data, and temporal dynamics to ensure that the expanded network accurately reflects the biological context of the target cellular phenotype.

The pathway identification and ranking process may involve determining potential connections from the candidate compounds to a given condition via the logic-based model by computing a ranked list of pathways through searching a directed protein interaction network and minimizing a cost function of the product of edge weights along each pathway. Each edge in the directed protein interaction network may be assigned a weight that reflects factors such as interaction confidence, experimental evidence, or biological relevance. The pathway search process may result in identification of multiple candidate drug pathways for each successfully mapped compound, representing potential mechanistic routes through which the compounds may modulate cellular processes related to cardiac hypertrophy or other conditions of interest.

As described above, the PathLinker algorithm may be utilized to identify top scoring directed pathways from drug targets to signaling network nodes. The PathLinker algorithm may implement pathway optimization methods that search through directed protein interaction networks to find routes connecting source nodes, representing drug targets, to destination nodes within the logic-based signaling network model. The algorithm may evaluate multiple potential pathways and rank them according to optimization criteria that consider network topology and interaction reliability. The cost function minimization process may involve calculating the product of edge weights along each potential pathway connecting drug targets to network nodes. Each edge in the directed protein interaction network may be assigned a weight that reflects factors such as interaction confidence, experimental evidence, or biological relevance. The PathLinker algorithm may identify pathways that minimize the cumulative cost function, thereby prioritizing routes with higher confidence interactions or shorter pathway lengths.

The pathway search process may result in identification of multiple candidate drug pathways for each successfully mapped compound. In some cases, analysis of candidate compounds may yield candidate drug pathways connecting compounds to the hypertrophy signaling network. These pathways may represent potential mechanistic routes through which the compounds may modulate cellular processes related to cardiac hypertrophy or other conditions of interest.

The inclusion of intermediate proteins in pathway predictions may reveal previously unrecognized connections between drug targets and cellular phenotypes. These intermediate proteins may serve as bridging components that link drug targets to established signaling network nodes through multi-step interaction cascades. The pathway ranking process may enable systematic comparison of different compounds and their predicted mechanisms of action, serving as a decision-support tool that guides resource allocation in drug discovery programs. The computational efficiency of the pathway identification process may enable analysis of large compound libraries and extensive protein interaction networks while maintaining reasonable computational performance.

The pathway ranking process may enable systematic comparison of different compounds and their predicted mechanisms of action. Compounds with higher-ranked pathways may be prioritized for experimental validation, while compounds with lower-ranked or less reliable pathways may be deprioritized or subjected to additional computational analysis. The ranking system may therefore serve as a decision-support tool that guides resource allocation in drug discovery programs.

The computational efficiency of the pathway identification process may enable analysis of large compound libraries and extensive protein interaction networks. The PathLinker implementation may handle networks containing thousands of proteins and interactions while maintaining reasonable computational performance. This scalability may support comprehensive drug discovery applications that require analysis of diverse compound collections against multiple target conditions or cellular phenotypes.

The process of creating expanded logic-based differential equation model variants may involve systematic integration of individual drug pathways into the base signaling network architecture. Each candidate drug pathway may be added as a separate model variant to enable independent evaluation of pathway contributions to drug effects. The expanded model variants may include additional nodes representing intermediate proteins identified through pathway mapping, along with the corresponding edges representing regulatory interactions between these proteins and existing network components. Each expanded model variant may be simulated to test the functional impact of the incorporated drug pathway on the cellular phenotype of interest through logic-based equations that account for drug binding properties and target modulation effects.

The expanded model variants may be utilized to predict drug efficacy across different experimental conditions and cellular contexts. The validated expanded models may simulate drug effects under various stimulus conditions that correspond to different disease states or experimental protocols. This capability may enable prediction of context-dependent drug efficacy and identification of optimal therapeutic applications for individual compounds.

The expanded network models may then be validated at step 110 against established experimental data to assess their functional accuracy and biological relevance. This validation process may involve systematically simulating the response of each expanded network variant to a comprehensive set of known perturbations and comparing the predicted results against previously published experimental data. The validation may utilize a curated database of experimental results that includes diverse perturbation conditions, such as gene knockdowns, protein overexpression studies, pharmacological interventions, and stimulus-response experiments. The system may calculate validation scores for each expanded network variant by comparing predicted network outputs against experimental measurements across multiple experimental conditions and cellular contexts. Network variants that maintain validation accuracy above a predetermined threshold (e.g., 75% accuracy, 80% accuracy, etc.) may be retained for further analysis and drug response simulation, while those that significantly reduce model performance below acceptable levels may be excluded from subsequent computational steps. The validation process may also include sensitivity analyses to determine the robustness of network predictions under varying parameter conditions and may incorporate confidence intervals or uncertainty quantification to assess the reliability of model predictions. Drug pathways that maintain model validation accuracy serve as quality control mechanisms that filter out pathways inconsistent with established network behavior, thereby improving the reliability of drug mechanism predictions and supporting drug repurposing applications by providing mechanistic validation for predicted off-target effects.

The model expansion and validation process may support drug repurposing applications by providing mechanistic validation for predicted off-target effects. Drugs that show validated pathway connections to cellular phenotypes different from their primary therapeutic targets may represent candidates for repurposing applications. The validation process may provide the mechanistic evidence needed to support experimental testing of these repurposing opportunities.

At step 112, the validated expanded networks may be used to simulate drug responses under various cellular conditions. The simulation process may implement logic-based differential equations that account for drug mechanisms of action, including agonist or antagonist effects and competitive or noncompetitive binding properties. These simulations may predict how drugs affect specific cellular phenotypes across different biochemical environments and stimulation conditions. The simulation framework may utilize established pharmacological models to represent drug-target interactions, incorporating parameters such as binding affinity, dissociation constants, and dose-response relationships. In some embodiments, the system may simulate drug effects by first establishing baseline network activity in the absence of drug treatment, then introducing the drug perturbation to identify steady-state changes in cellular responses. The simulation process may account for context-dependent drug efficacy by modeling responses across multiple biochemical environments, including different hypertrophic stimuli, varying concentrations of signaling molecules, and diverse cellular stress conditions. The logic-based differential equation framework may enable semi-quantitative predictions of network dynamics, allowing the system to model temporal changes in protein activities and gene expression levels following drug treatment. Additionally, the simulation may incorporate dose-dependent effects by modeling drug concentration gradients and their corresponding impacts on target protein activities. The system may generate predictions for multiple cellular readouts simultaneously, including morphological changes, protein expression patterns, and functional cellular responses, thereby providing comprehensive insights into drug mechanisms of action across different biological contexts.

Drug mechanisms of action may be implemented using logic-based equations that account for various mechanisms of drug action and drug binding properties. The logic-based equations may incorporate mathematical representations of how drugs interact with their molecular targets and how these interactions affect cellular signaling networks. These equations may provide a framework for simulating drug effects within the context of complex signaling pathway models.

The logic-based equations may account for different mechanisms of drug action, including agonist and antagonist mechanisms. Agonist mechanisms may involve drug binding that enhances or activates the function of the target protein, while antagonist mechanisms may involve drug binding that inhibits or blocks the function of the target protein. The equations may incorporate parameters that distinguish between these different modes of action and predict their respective effects on downstream signaling processes.

By way of example, drug binding properties may be represented within the logic-based equations through parameters that describe competitive and noncompetitive binding interactions. Competitive binding may occur when a drug competes with natural ligands for the same binding site on the target protein, while noncompetitive binding may occur when a drug binds to a different site and affects protein function through allosteric mechanisms. The equations may incorporate binding kinetics and affinity parameters that reflect these different binding modes. The binding properties may determine how the upstream signal is shifted by the drug dose within the logic-based simulation framework. Competitive binding interactions may result in dose-dependent shifts in the effective concentration of natural ligands, while noncompetitive binding interactions may result in dose-dependent changes in the maximum response or sensitivity of the target protein. The logic-based equations may calculate these signal shifts based on drug concentration, binding affinity, and competition parameters.

The drug action mechanism may therefore determine whether the target node is upregulated or downregulated within the signaling network simulation. Agonist drug actions may result in upregulation of the target node activity, leading to enhanced signal propagation through downstream pathways. Antagonist drug actions may result in downregulation of the target node activity, leading to reduced signal propagation or pathway inhibition. The logic-based equations may implement these regulatory effects through mathematical functions that modify node activity levels based on drug presence and concentration.

Each simulation may be run first without drug input to establish baseline activity levels within the signaling network. The baseline simulation may provide reference values for node activities and pathway outputs under normal physiological conditions. Subsequently, simulations may be performed with the appropriate drug parameters to identify steady-state changes in drug response compared to the baseline condition.

Context-dependent drug efficacy may be predicted depending on the hypertrophic stimulus used in the experimental or clinical setting. Different hypertrophic stimuli may activate distinct subsets of signaling pathways within the network model, leading to different patterns of node activities and regulatory interactions. The logic-based equations may account for these stimulus-specific network states when predicting drug effects. Context-dependent efficacy predictions may arise from the interaction between drug mechanisms and the specific signaling pathways activated by different hypertrophic stimuli. A drug that targets a pathway component may show different efficacy levels depending on whether that component is activated or plays a regulatory role under specific stimulus conditions. The logic-based simulation framework may capture these context-dependent interactions through dynamic modeling of network responses.

The simulation process may evaluate drug efficacy across multiple biochemical environments that represent different hypertrophic conditions or disease states. Each biochemical environment may be characterized by different patterns of pathway activation, protein expression levels, or regulatory interactions. The logic-based equations may predict how drug effects vary across these different environments, providing insights into potential therapeutic applications and patient stratification strategies.

The mechanistic simulation approach may enable prediction of drug effects under various experimental conditions that correspond to different cellular contexts or disease models. The logic-based equations may incorporate parameters that reflect different experimental conditions, such as the presence of specific growth factors, cytokines, or other signaling molecules that may influence drug efficacy. This capability may support experimental design and interpretation of drug screening results across diverse testing conditions.

The method may proceed to step 114 with mechanistic subnetwork analysis to identify the specific pathways mediating drug responses and provide detailed mechanistic understanding of drug action. This analysis may involve simulating global network responses to drug treatment in the presence of relevant stimuli to identify drug-responsive nodes that exhibit significant changes in activity levels following drug perturbation. The system may then perform systematic node knockdown experiments in silico by individually suppressing each network node and measuring the influence of that node on drug-induced changes in the target cellular phenotype, thereby identifying pathway-regulating nodes that control drug efficacy. The mechanistic subnetwork analysis may determine the intersection of drug-responsive nodes with pathway-regulating nodes to create focused subnetworks that capture the essential molecular mechanisms underlying drug action. The resulting mechanistic subnetworks may provide detailed insights into how drugs exert their effects on cellular phenotypes, including identification of key regulatory proteins, critical pathway branches, and potential points of therapeutic intervention. In some embodiments, the mechanistic subnetwork analysis may generate confidence scores for pathway components based on their regulatory influence and drug responsiveness, allowing prioritization of the most relevant mechanistic elements. The system may also perform sensitivity analysis on the mechanistic subnetworks to assess the robustness of predicted drug mechanisms under varying biological conditions and parameter uncertainties. Additionally, the mechanistic subnetwork analysis may identify potential off-target effects by revealing unexpected pathway connections and may suggest combination therapy opportunities by highlighting complementary or synergistic pathway interactions.

By way of example, the mechanistic subnetwork analysis may provide a method for explaining drug response and supporting validation of drug use for a given patient or patient population. The mechanistic subnetwork method may identify specific network components that mediate drug effects by systematically analyzing how individual network nodes contribute to observed drug responses. This analysis may enable identification of the pathways through which drugs achieve their therapeutic effects and may provide mechanistic insights that support clinical decision-making.

The mechanistic subnetwork method may begin by simulating the global response of the network to drug treatment in the presence of a hypertrophic, or other suitable, stimulus. This global simulation may identify nodes within the signaling network that exhibit changes in activity or expression levels in response to drug treatment. These drug-responsive nodes may represent network components that are directly or indirectly affected by the drug perturbation and may include both immediate targets and downstream effectors.

The identification of drug-responsive nodes may involve comparing network simulations performed with and without drug treatment under identical stimulus conditions. Nodes that show statistically significant changes in activity levels between the drug-treated and control conditions may be classified as drug-responsive. The magnitude and direction of these changes may provide information about whether specific network components are activated or inhibited by drug treatment.

Following identification of drug-responsive nodes, the mechanistic subnetwork method may simulate knockdown of each individual node within the signaling network. The knockdown simulations may involve reducing the activity or expression level of each node to minimal levels while maintaining normal activity levels for all other network components. These simulations may be performed under both drug-treated and control conditions to assess how individual node perturbations affect the overall drug response. The knockdown simulations may enable measurement of the influence of each node on drug-induced changes in the cellular phenotype(s) of interest (e.g., cardiomyocyte hypertrophy). Nodes whose knockdown significantly alters the magnitude or direction of the drug response may be classified as regulating nodes. These regulating nodes may represent network components that play roles in mediating or modulating the effects of the drug on the cellular phenotype.

The mechanistic subnetwork may be defined as the intersection of the regulating nodes with the responsive nodes identified through the global network simulation. This intersection may identify network components that both respond to drug treatment and contribute to the drug's effects on the cellular phenotype. The mechanistic subnetwork may therefore represent the specific pathways and molecular components through which the drug achieves its therapeutic effects.

The mechanistic subnetwork analysis may map the pathways mediating drug activity by identifying the regulatory connections between nodes within the mechanistic subnetwork. These pathways may represent the sequence of molecular interactions through which drug effects propagate from the initial target to the final cellular outcome. The pathway mapping may reveal both direct and indirect mechanisms through which drugs modulate cellular processes.

The drug discovery process may utilize the mechanistic subnetwork analysis to support validation of drug use for specific patient populations. The mechanistic subnetwork may provide insights into the molecular requirements for drug efficacy, such as the expression levels or activity states of specific proteins that mediate drug effects. This information may enable identification of biomarkers that predict drug responsiveness in individual patients.

Advantageously, the mechanistic subnetwork method may enable prediction of drug efficacy across different patient populations by analyzing how variations in network component expression or activity may affect drug response pathways. Patients with altered expression or function of proteins within the mechanistic subnetwork may exhibit different responses to drug treatment compared to patients with normal network function. This analysis may support patient stratification strategies that optimize therapeutic outcomes. The mechanistic subnetwork analysis may be integrated with other computational methods, such as pathway optimization and logic-based simulation, to provide a complete framework for understanding drug mechanisms.

The mechanistic subnetwork analysis may provide validation support for drug repurposing applications by revealing the molecular basis for off-target therapeutic effects. Drugs that achieve beneficial effects through pathways different from their primary mechanisms may be identified through mechanistic subnetwork analysis of their effects on alternative cellular phenotypes. This analysis may provide the mechanistic rationale needed to support regulatory approval for new therapeutic indications. Additionally or alternatively, the mechanistic subnetwork method may enable identification of combination therapy opportunities by analyzing how different drugs affect overlapping or complementary network pathways. Drugs that target different components within the same mechanistic subnetwork may exhibit synergistic effects, while drugs that target independent subnetworks may provide additive therapeutic benefits. The mechanistic subnetwork analysis may therefore support rational design of combination therapy regimens.

At step 116, the system may generate comprehensive outputs that provide actionable insights for drug discovery and therapeutic applications. The outputs may include ranked lists of candidate compounds with their predicted efficacy scores, detailed mechanistic pathway maps showing how each drug modulates the target cellular phenotype, and confidence metrics associated with each prediction. The system may generate visual representations of the mechanistic subnetworks, highlighting key regulatory nodes and pathway interactions that mediate drug responses. Additionally, the outputs may include comparative analyses showing how different drugs affect the same cellular phenotype through distinct molecular mechanisms, enabling researchers to identify potential combination therapies or alternative treatment strategies. The system may also provide validation recommendations, suggesting specific experimental approaches to confirm the predicted drug mechanisms in laboratory settings. In some embodiments, the outputs may be formatted for integration with existing drug discovery pipelines, including compatibility with pharmaceutical databases, regulatory submission requirements, and clinical trial design protocols. The generated outputs may be customized based on user preferences, therapeutic area focus, and intended application, whether for academic research, pharmaceutical development, or clinical decision support.

In some cases, the ranked list of pathways generated in step 108 may be output to a user for further analysis and drug discovery applications. The output may include pathway information such as the sequence of protein interactions, confidence scores for individual pathway components, and overall pathway rankings based on the cost function optimization. Users may utilize this ranked pathway information to prioritize compounds for experimental testing, design mechanistic studies, or identify potential drug repurposing opportunities.

To address the challenges of mapping and mechanistically understanding drug responses from phenotypic screens, a logic-based predictor of drug pathways (LogiRx) was developed. LogiRx maps from drug-target and protein interaction databases to curated logic-based network models of cell phenotype. LogiRx was used to prioritize seven antihypertrophic drugs identified in a previous cell-based screen, providing mechanistic insight into how two drugs attenuate hypertrophy via “off-target” pathways. LogiRx predictions were experimentally validated in cultured cardiomyocytes, mice, and in two patient databases of electronic health records. Together, these studies validate the LogiRx method and provide mechanistic insights into how escitalopram (Lexapro) and other drugs modulate cardiomyocyte hypertrophy.

### Linking Drug Targets to Logic-Based Signaling Network Using LogiRx.

A published logic-based differential equation model of the cardiomyocyte hypertrophy signaling network was used. This literature-based network of proteins and genes is composed of 107 nodes (proteins or mRNAs) and 193 edges, validated against 450 experiments across the literature with 77% accuracy. The model correctly predicted the antihypertrophic effect of 28 out of 32 drug responses from a previous experimental screen.

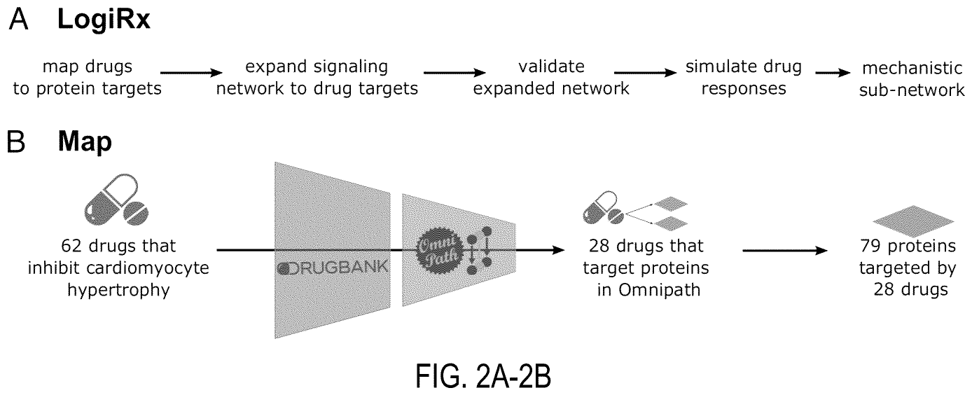

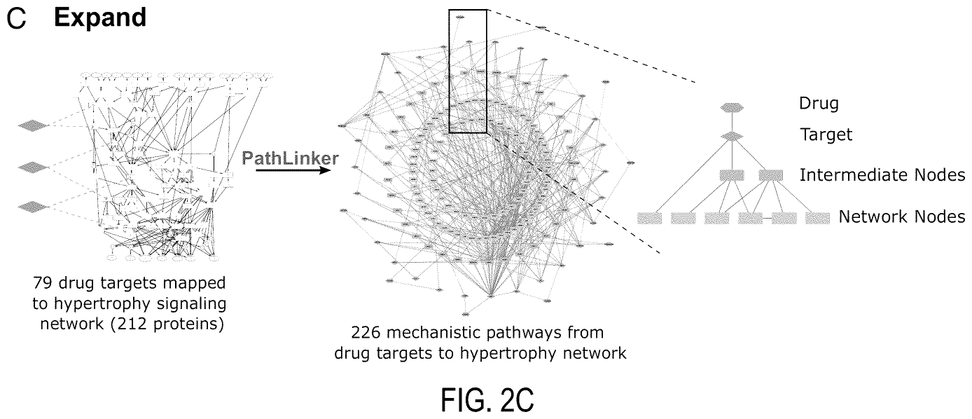

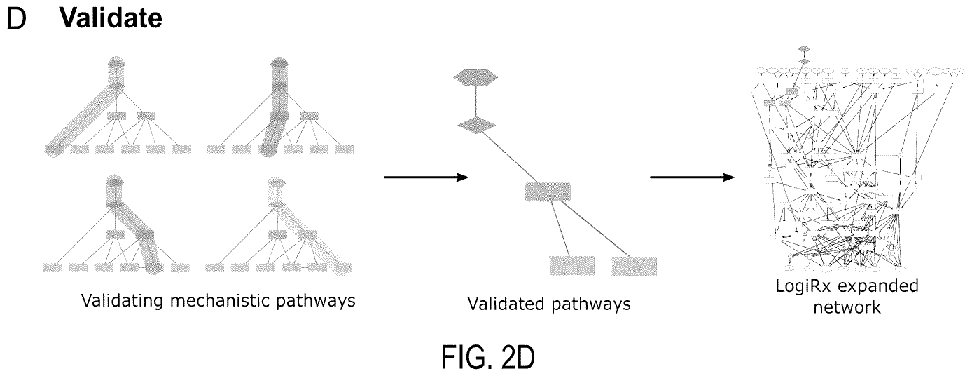

Hits from a previous screen for compounds that inhibit cardiomyocyte hypertrophy were first identified, with protein targets identified using the drug-target database DrugBank (FIG. 2A). LogiRx identified top scoring directed pathways from drug targets to the hypertrophy signaling network nodes through OmniPath directed interactions using the PathLinker algorithm in Cytoscape (FIG. 2B). Top scoring candidate drug pathways were added to a LogiRx-expanded logic-based network model using Netflux.

### Literature Validation of LogiRx-Expanded Models.

The LogiRx-expanded logic-based differential equation model was simulated to test the functional impact of the drug pathways on cardiomyocyte hypertrophy. Each pathway was individually added to the signaling network model to create expanded logic-based differential equation model variants. Each drug-pathway-expanded model was simulated to predict the semiquantitative network dynamics in response to drug. To assess the functional validity of the LogiRx-identified drug pathways, their performance was validated against 450 literature experiments previously used to validate the hypertrophy signaling model (FIG. 2C).

### Simulating Drug Mechanisms of Action.

Drug mechanisms of action were implemented using logic-based equations as described previously, accounting for mechanisms of drug action (agonist or antagonist) and drug binding (competitive or noncompetitive). The binding properties determine how the upstream signal is shifted by the drug dose, while the drug action determines whether the target node is upregulated (agonist) or downregulated (antagonist). Each simulation was run first without drug input to establish baseline activity and then with the appropriate drug to identify steady-state changes in drug response.

### Mechanistic Subnetwork Analysis.

To identify pathways mediating drug response, the mechanistic subnetwork method was utilized. The global response of the network to the drug in the presence of hypertrophic stimulus was simulated to identify “drug-responsive nodes.” Knockdown of each node was then simulated and that node's influence on drug-induced change in cardiomyocyte hypertrophy was measured, referring to these nodes as “regulating nodes.” The intersection of the regulating nodes with responsive nodes results in a mechanistic subnetwork that maps the pathways mediating drug activity. Thus, LogiRx combines drug-target and protein interaction databases, path optimization, and logic-based network simulations to obtain mechanistic subnetworks that explain drug response.

### Experimental Validation in Cardiomyocytes.

Neonatal cardiomyocytes were isolated using the NeoMyts kit from Cellutron as described previously. Cardiomyocytes were cultured with serum for 24 h in 96-well microplates, followed by a 24 h serum starve. Cardiomyocytes were then treated with one of two hypertrophic stimuli (10 μM phenylephrine, 5 ng/ml transforming growth factor β), 10% FBS (positive control), or serum-free media alone (negative control). At the same time, cells were treated with specified concentrations of the appropriate compound (escitalopram, mifepristone, paroxetine, sarpogrelate hydrochloride, or AL082D06) for 48 h.

To prepare for immunofluorescent imaging, cardiomyocytes were first fixed with 4% paraformaldehyde for 20 min and then permeabilized with 0.1% Triton-X for 15 min. Cardiomyocytes were blocked with 1% bovine serum albumin in PBS for 1 h, then treated with mouse anti-α-actinin primary antibody (Sigma-Aldrich Cat #A7811, RRID:AB_476766) at a concentration of 1:200 overnight. Cardiomyocytes were blocked with 5% goat serum in PBS for 1 h, then Alexa Fluor-568-conjugated goat anti-mouse secondary antibody (Thermo Fisher Scientific Cat #A11031, RRID:AB_144696) at a concentration of 1:200 was applied for 1 h. The cells were stained with DAPI prior to imaging.

High-content imaging was performed on the stained cardiomyocytes using an Operetta CLS High Content Analysis System. These images were processed using CellProfiler using a cellular segmentation algorithm developed previously and validated to within 5% of two independent manual segmentations. Median cell area was used as a representative measure of the cell population in each well, and cells with undetectable cytoplasm were not counted.

Total protein was isolated from neonatal rat ventricular myocytes (NRVM) using RIPA lysis buffer (25 mM Tris pH 7.6, 150 mM NaCl, 1% NP-40, 1% sodium deoxycholate, and 0.1% SDS) supplemented with Halt™ protease and phosphatase inhibitor cocktail (ThermoFisher 78442). Proteins were separated using SDS-PAGE and transferred onto nitrocellulose membrane (0.45 μm, Thermo Scientific 88018). Total protein was first evaluated using LI-COR Revert™ 520 Total Protein Stain (926-10021), followed by blotting with antibodies directed against phosphorylated protein kinase D (1:1,000; CST 2051) and glyceraldehyde phosphate dehydrogenase (1:5,000; Proteintech 60004-1-Ig) overnight at 4° C. in Intercept® (TBS) Blocking Buffer (LI-COR 927-60001) containing 0.2% Tween-20. Following incubation with primary antibodies, blots were rinsed and probed with appropriate IRDye® secondary antibodies (LI-COR). Protein bands were visualized using a LI-COR Odyssey XF scanner.

### Angiotensin II/Phenylephrine Infusion in Mice.

All animal procedures were performed in accordance with the Institutional Animal Care and Use Committee at the University of Colorado Anschutz Medical Campus. Ten-week-old male C57BL/6J were purchased from The Jackson Laboratory (Strain #000664). Alzet miniosmotic pumps (Model 2004), or mock pumps for Sham animals, were implanted in a subcutaneous pocket created at the suprascapular region under isoflurane anesthesia. Osmotic pumps were prepared to deliver 1.5 μg/kg/d Angiotensin II (Bachem H-1705.0100) and 50 μg/g/d (R)-(−)-Phenylephrine hydrochloride (Sigma P6126) to induce systemic hypertension, cardiac hypertrophy, and fibrosis. Beginning 24 h postosmotic pump implantation, animals were randomly divided into groups receiving 10 mg/kg/d escitalopram oxalate (Selleckchem S4064) in 10% DMSO dissolved in 10% (2-hydroxypropyl) β-cyclodextrin (Sigma H107) in water, or vehicle control, for 14 d.

### Echocardiographic Assessment of Cardiac Structure and Function.

Serial transthoracic echocardiographic and Doppler analysis were performed using the VisualSonics Vevo F2 instrument. Animals were anesthetized with 2% isoflurane, hair on the chest was removed using a chemical depilatory, and body temperature maintained at 37° C. Isoflurane was maintained at 1.5% throughout the procedure to ensure a consistent plane of anesthesia across subjects. Parasternal long axis (PSLAX) and parasternal short axis (SAX) views of the left ventricle were obtained; SAX two-dimensional views of the LV at the level of the papillary muscle were used to acquire M-mode images. Anterior and posterior LV wall thickness and internal diameters were measured in systole and diastole using these M-mode images. Mitral inflow Doppler signals and myocardial tissue movement of the mitral annulus were obtained to calculate the ratio of early and active filling waves of blood flow through the mitral valve and ventricular tissue velocity to assess cardiac diastolic function. Speckle-tracking strain analyses were acquired from PSLAX B-mode videos. All echocardiographic measurements and analyses were averaged from at least four cardiac cycles and analyses were performed in a blinded manner by a dedicated small animal echocardiography team.

### Tissue Procurement.

At the study endpoint, animals were killed by exsanguination, and hearts were excised and placed in ice-cold saline. Right ventricular (RV) tissue was dissected from the LV by cutting along the septum and the outer wall of the LV, and all parts were weighed. 50 mg base and apical biopsies of the LV were flash-frozen in liquid nitrogen for subsequent biochemical analysis. A cross-section of the LV at the level of the papillary muscles was fixed in 4% PFA in PBS overnight at 4° C. followed by preservation in a 30% sucrose solution and embedding in OCT.

### Histological Analyses.

Frozen tissue sections were cut at a thickness of 10 μm. To evaluate total ventricular fibrotic area, cardiac sections were stained with Picrosirius red dye (0.1% Direct Red 80 in saturated picric acid solution) for 1 h at room temperature. Whole-heart images were acquired using the image stitching feature on a Keyence BZ-X710 All-in-One Fluorescence Microscope. Fibrotic area was determined by quantifying the ratio of positively stained (red) pixels to the total pixel number of each section (reported as percent collagen content) using ImageJ. In addition, individual cardiomyocyte hypertrophy was assessed by staining cardiac sections with Alexa Fluor 647 wheat germ agglutinin (ThermoFisher W32466) according to manufacturer instructions. Briefly, sections were incubated in a 5.0 μg/mL WGA conjugate solution in HBSS for 10 min at room temperature, followed by mounting in ProLong™ Glass Antifade Mountant (ThermoFisher P36980). Images were acquired at 40× magnification with the left ventricular free wall using the Keyence BZ-X710 microscope, and individual cardiomyocyte area was quantified using ImageJ.

Patient Outcomes Associated with Drug Treatments.

To examine whether drugs were correlated with a reduction in cardiac adverse events associated with hypertrophy, data from two separate clinical databases were analyzed. As the first patient cohort, data were collected on Nov. 30, 2023, from the FDA's Adverse Event Reporting System using the AERSMine tool. The Adverse Event Reporting System is a multicohort database containing 20 million reports of adverse events from healthcare providers and consumers across the United States. As a second patient cohort, on Jan. 2, 2024 data were collected from the University of Virginia Health System Network using the TriNetX tool, which provided access to electronic medical records from approximately 1.7 million patients.

This retrospective study is exempt from informed consent. The data reviewed are a secondary analysis of existing data, do not involve intervention or interaction with human subjects, and are deidentified per the deidentification standard defined in Section § 164.514 (a) of the HIPAA Privacy Rule. The process by which the data are deidentified is attested to through a formal determination by a qualified expert as defined in Section § 164.514 (b)(1) of the HIPAA Privacy Rule. This formal determination by a qualified expert was refreshed on December 2020.

### Statistics.

All data are presented as the mean±SEM. Analysis of experimental conditions considered two distinct cardiomyocyte isolations and multiple conditions, with multiple wells in each experiment. For this reason, a two-way ANOVA followed by Dunnett's test for multiple comparisons was selected for statistical calculation. In vivo data were analyzed by ordinary one-way ANOVA followed by Tukey's multiple comparisons test. GraphPad Prism 9 was used for statistical analysis. For all analyses, a P-value <0.05 was considered statistically significant.

### Identified Drug Pathways Driving Cardiomyocyte Hypertrophy.

Previously, a microscopy-based screen for drugs that decreased hypertrophy of cardiomyocytes in the presence of phenylephrine was performed. In that study, 94 of 3,241 small molecule compounds decreased both cell area and ANP protein expression by at least 70% and did not exhibit cardiotoxicity. In a previous study, it was identified that the hypertrophy signaling model was directly targeted by 32 of these 94 drugs and that the model could mechanistically predict reduction in hypertrophy with 88% (28 of 32) prediction accuracy. The remaining 62 of 94 compounds do not target this previous hypertrophy model and presumably inhibit cardiomyocyte hypertrophy through less-characterized mechanisms. Therefore, in this study, pathways linking the remaining 62 compounds to hypertrophy were sought to be identified. To identify new drug pathways, a logic-based network predictor of drug pathways (LogiRx) was developed that identifies and simulates molecular pathways that mediate how drugs regulate cell phenotype.

As a first application, LogiRx was employed to identify proteins that are directly targeted by the 62 antihypertrophic compounds from the prior screen. LogiRx analysis mapped 28 of the 62 compounds to 79 protein targets in the OmniPath protein interaction database (FIG. 2A). 11 of 28 compounds were successfully mapped to the hypertrophy network via 214 candidate drug pathways (FIG. 2B). These pathways link 11 drugs and their drug targets to 89 proteins in the network, covering 42% of the network. Most of the candidate drug pathways (76%) connect the drug target to the network via an intermediate protein that is not in the original network model, demonstrating the value of pathway inference. Together, the 214 candidate drug-target-network pathways predict mechanisms that may help explain the experimentally demonstrated antihypertrophic effect of these 11 compounds.

### Logic-Based Network Expansion to Predict How Drugs Regulate Hypertrophy.

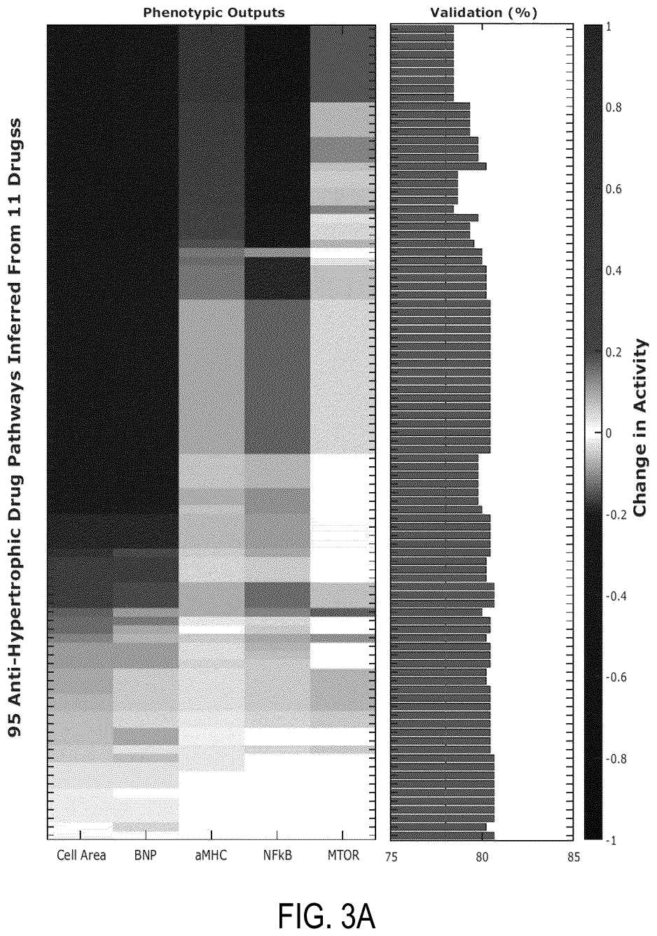

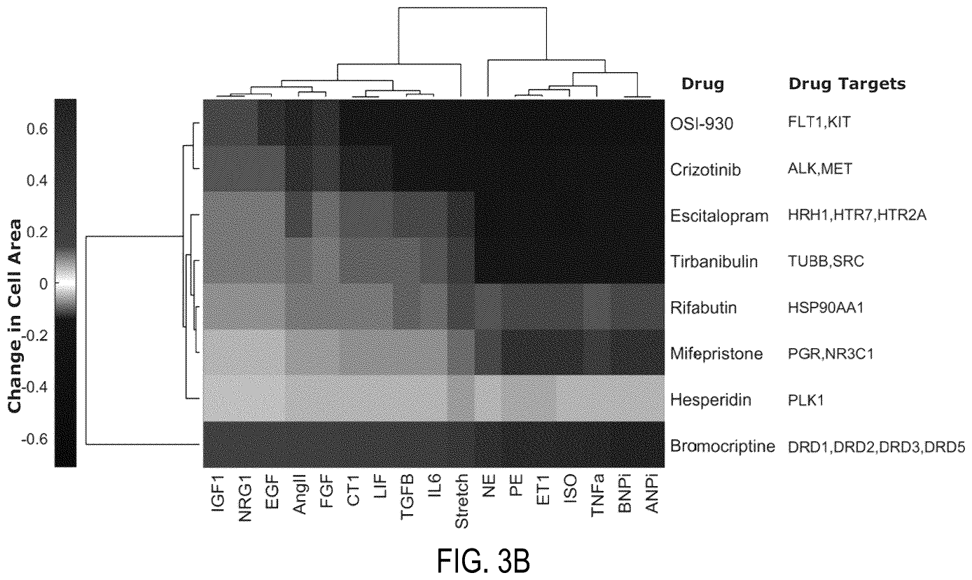

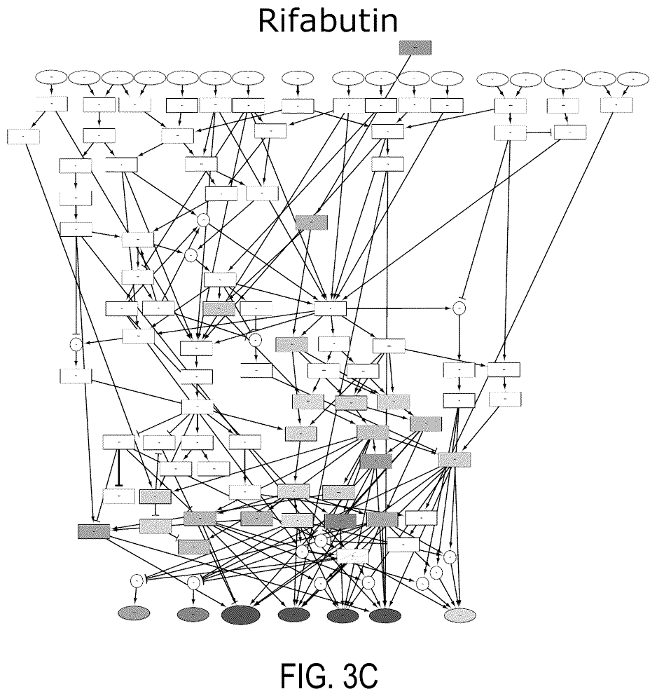

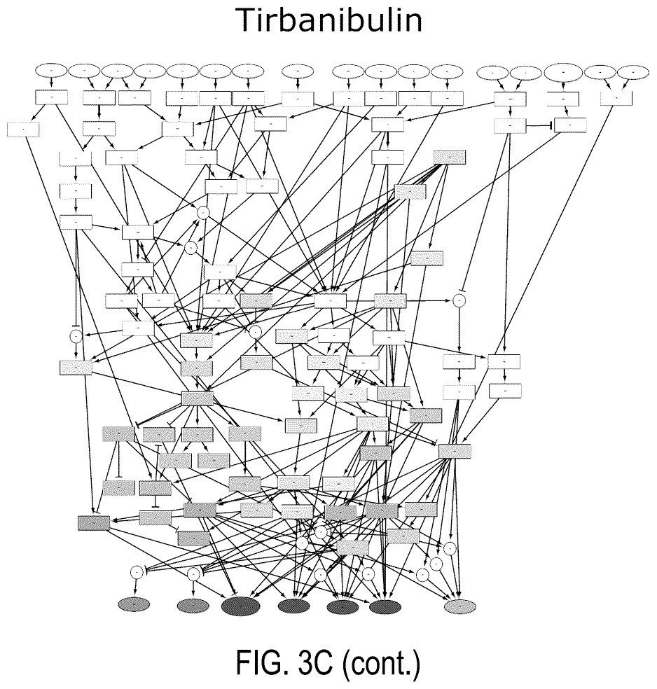

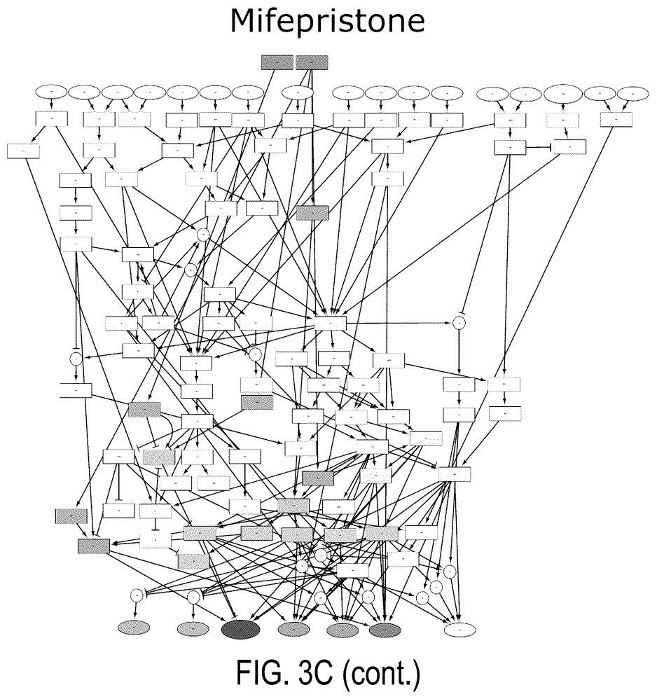

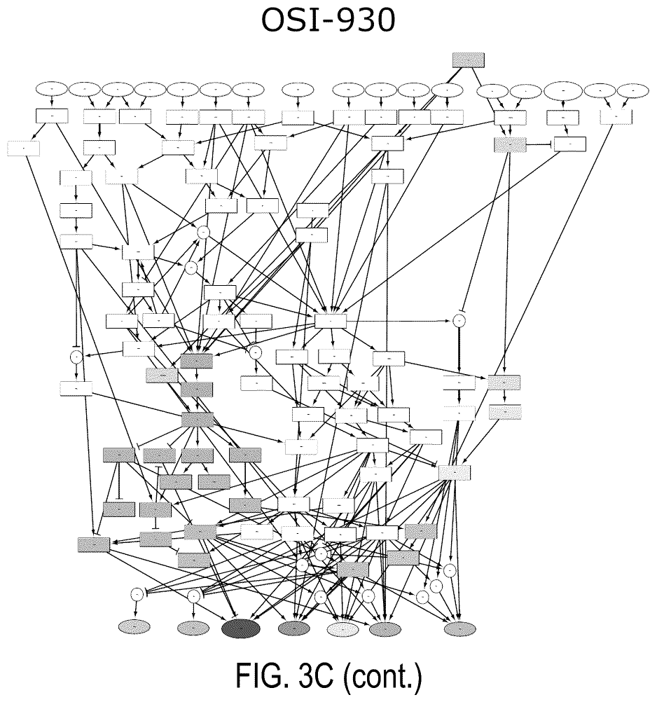

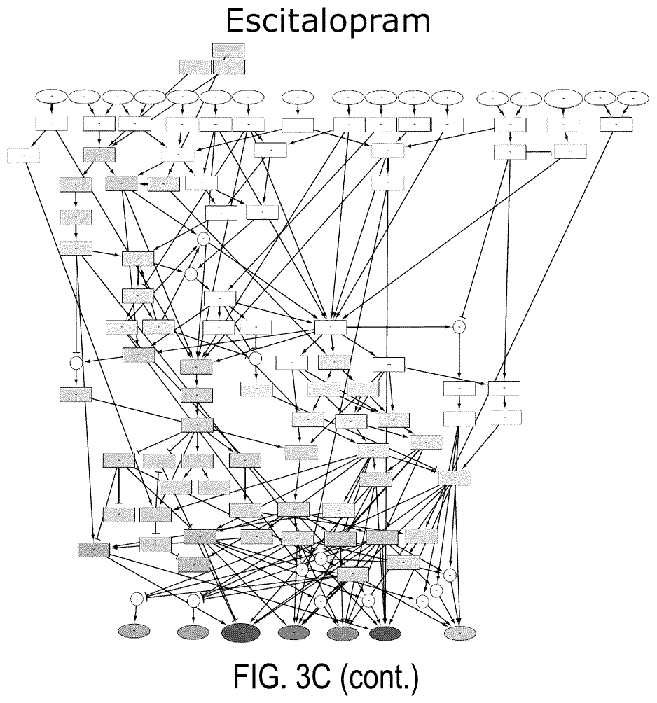

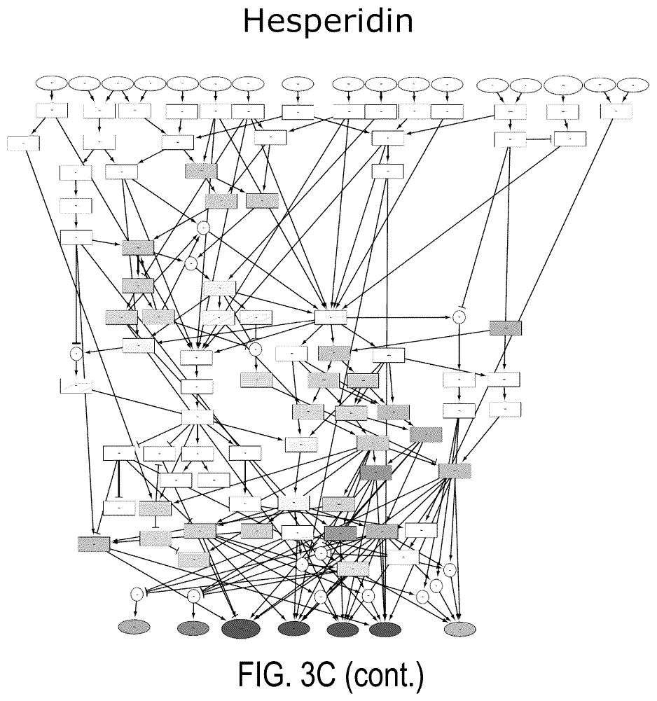

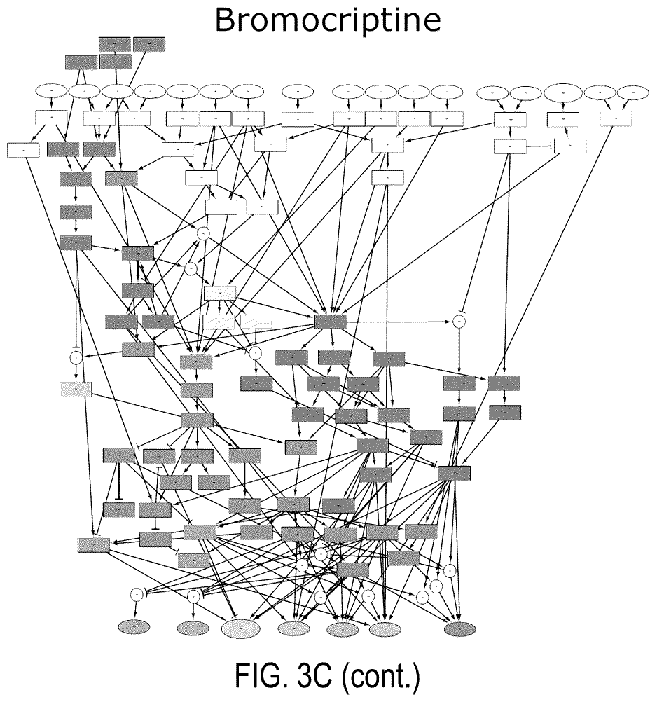

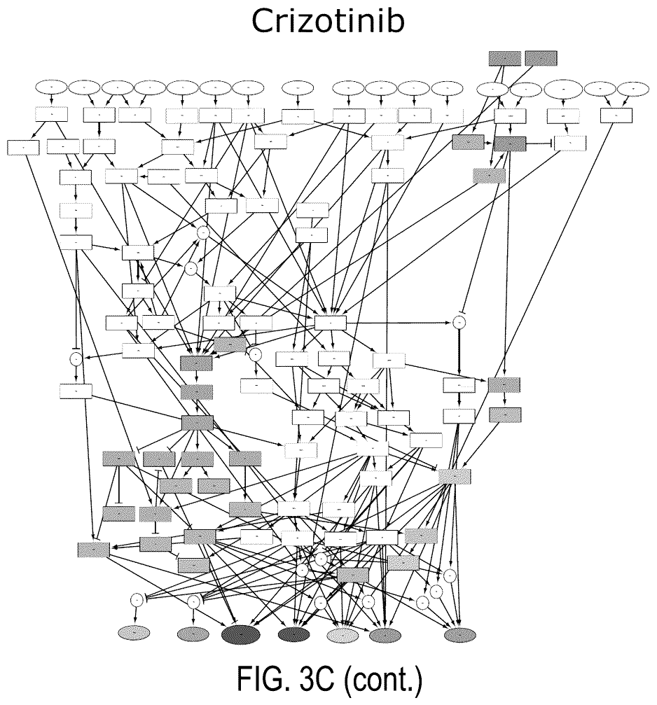

To predict the impact of these drug pathways on cardiomyocyte hypertrophy, logic-based differential equation model variants were created from LogiRx that each simulate the effect of incorporating individual pathway (FIG. 2C). Each model variant was tested by simulating inhibition of the drug target in the presence of hypertrophic agonist phenylephrine. Most drug pathways were predicted to strongly inhibit hypertrophy (FIG. 3A). However, some drug pathways reduced the model accuracy when tested against a previous set of 450 experiments, indicating that they are not fully compatible with the mechanisms established in the validated network model (FIG. 3A).

Drug pathways corresponding to 8 of the 11 drugs were predicted to inhibit phenylephrine-induced hypertrophy while maintaining model accuracy above 78%. LogiRx-expanded drug models were developed combining all relevant validated pathways for each of the eight drugs. The hypertrophic effect of each drug was simulated using a pharmacological inhibition model based on known drug-target binding mechanisms. Seven of these eight drugs were predicted to be antihypertrophic, with context-dependent efficacy depending on the hypertrophic stimulus (FIG. 3B). 7 of 8 drugs regulated hypertrophy via multiple protein targets.

Seven drugs were predicted by the LogiRx-expanded network model to inhibit hypertrophy-OSI-930, crizotinib, escitalopram, tirbanibulin, rifabutin, mifepristone, and hesperidin, representing a variety of drug targets and clinical applications. OSI-930 is an FLT1 and KIT inhibitor that is designed to target cancer cell proliferation in solid tumors. Crizotinib, an ALK and MET inhibitor, is used to treat non-small cell lung cancer but has known cardiac toxicity. Escitalopram targets SERT and is used to treat depression and anxiety. Tirbanibulin targets TUBB and SRC to treat actinic keratosis and is being investigated as a potential treatment for acute myeloid leukemia. Rifabutin inhibits HSP90AA1 and is an antibiotic preventing Mycobacterium avium complex in HIV patients. Mifepristone, which targets both PGR and NR3C1, is commonly used as an abortifacient, but is additionally used to treat diabetic patients with Cushing's syndrome. Hesperidin is a flavonoid and natural supplement currently investigated for anti-inflammatory benefits. Similar to their target diversity, these eight drugs were predicted to inhibit cardiomyocyte hypertrophy through distinct areas of the hypertrophy network (FIG. 3C).

Experiments in Cardiomyocytes Validate that Escitalopram and Mifepristone Inhibit Hypertrophy.

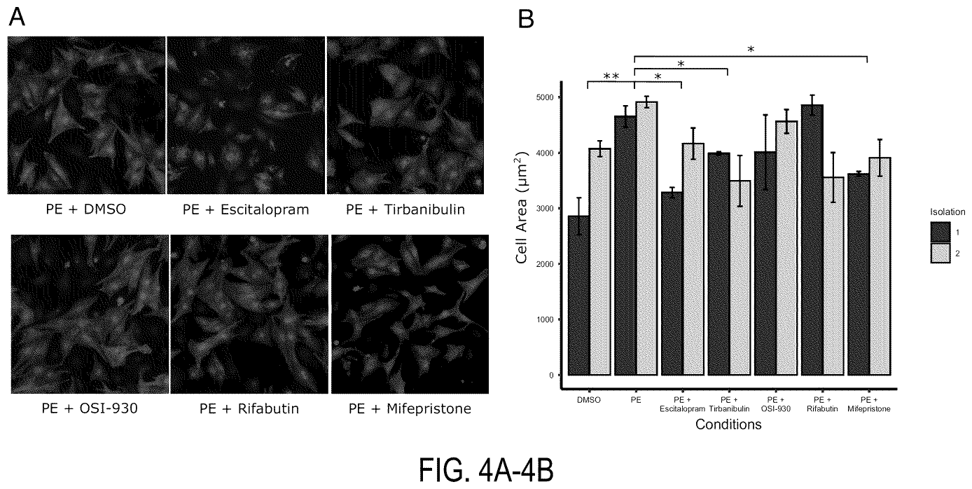

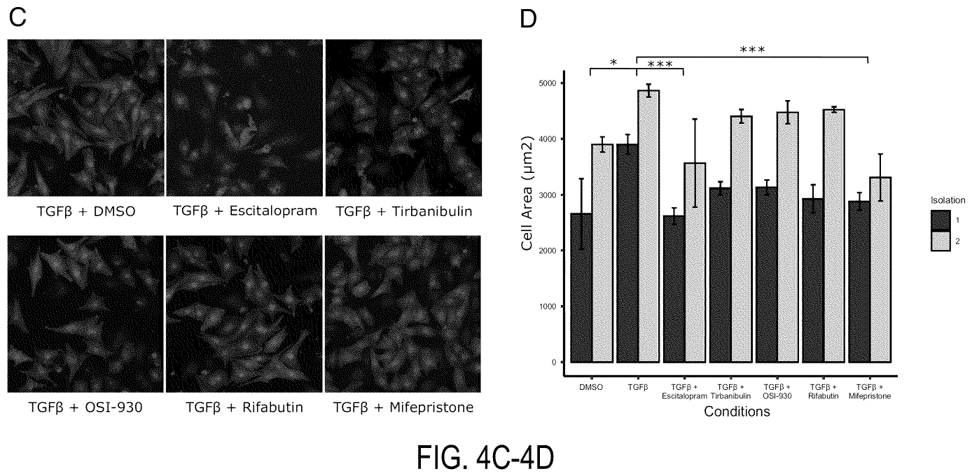

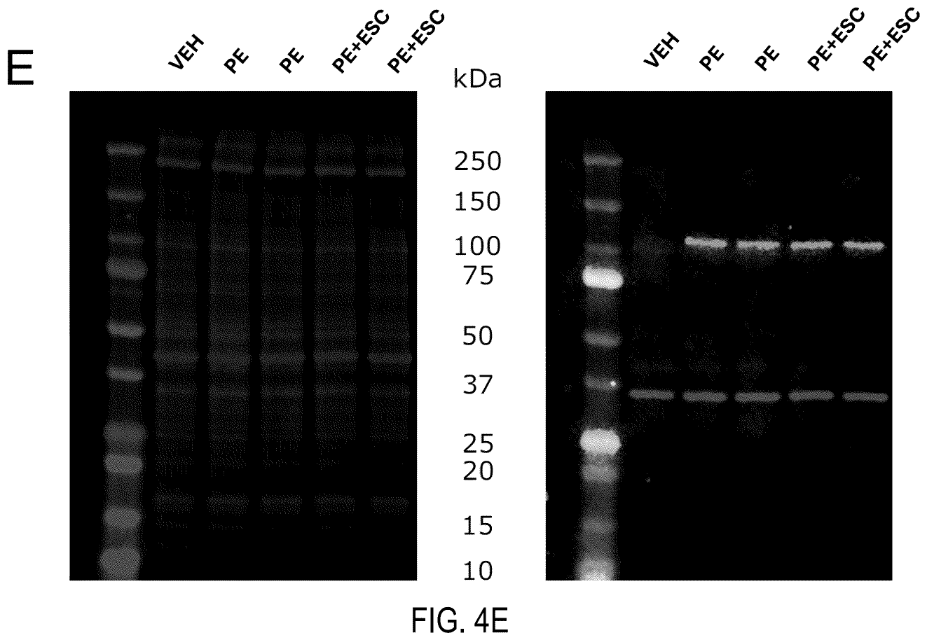

Escitalopram, tirbanibulin, mifepristone, OSI-930, and rifabutin were further prioritized for experimental validation based on their relative cardiac safety and approval status. Neonatal rat cardiomyocytes were treated with PE along with each of these five drugs for 48 h. Consistent with the LogiRx model predictions, tirbanibulin, escitalopram, and mifepristone inhibited PE-induced cardiomyocyte hypertrophy (FIGS. 4A and B). In contrast, OSI-930 and rifabutin did not inhibit hypertrophy even at increased concentrations.

While the network analysis in FIG. 3C predicted that escitalopram would work via downstream hypertrophic pathways that are relatively context-independent, an alternative hypothesis may suggest that escitalopram inhibits alpha-1 adrenergic receptor (α1AR) directly in response to phenylephrine. This hypothesis was tested by introducing an alternative hypertrophic stimulus TGFβ, as well as directly measuring phosphorylation of protein kinase D (PKD), which is proximal to the al adrenergic receptor in cardiomyocytes. TGFβ-induced hypertrophy was inhibited by both escitalopram and mifepristone (FIGS. 4 C and D). Further, while a robust increase in phospho-PKD levels in cardiomyocytes treated with phenylephrine stimulation was observed, no reduction in PKD phosphorylation was observed in cardiomyocytes cotreated with escitalopram (FIG. 4E). Together, these data indicate that escitalopram antagonizes hypertrophy through downstream mechanisms distinct from blockade of the α1AR.

To identify how mifepristone inhibits cardiomyocyte hypertrophy, mechanistic subnetwork analysis was performed. Although the LogiRx network model included drug pathways for mifepristone via both progesterone receptors and glucocorticoid receptors, LogiRx predicted that mifepristone inhibits cardiomyocyte hypertrophy predominantly via inhibition of the glucocorticoid receptor via LCK/ERK and CEBPβ. It was experimentally validated that the glucocorticoid receptor antagonist AL082D06 phenocopied the effects of mifepristone by preventing hypertrophy with PE or TGFβ.

Escitalopram Inhibits Hypertrophy Through Serotonin Receptor and Not Transporter.

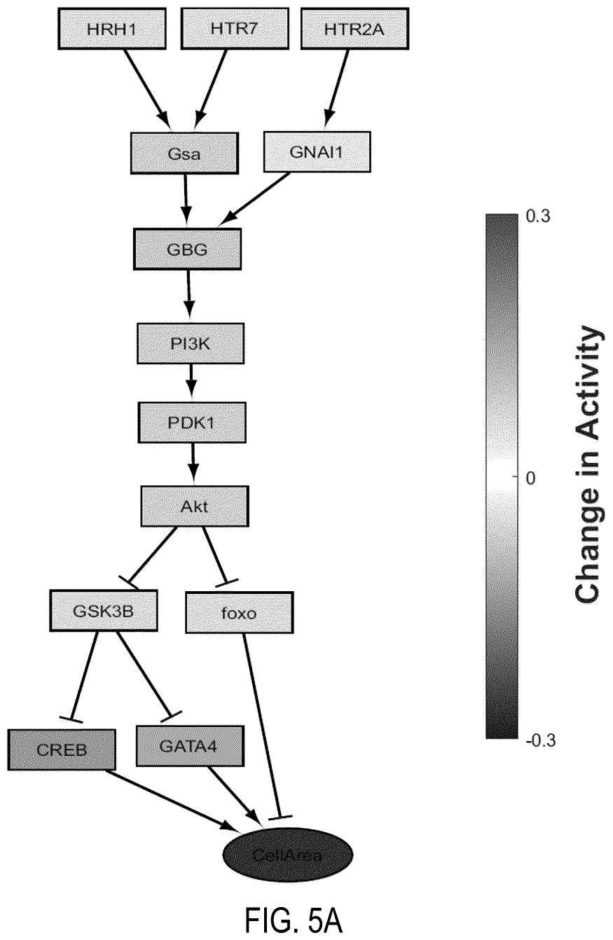

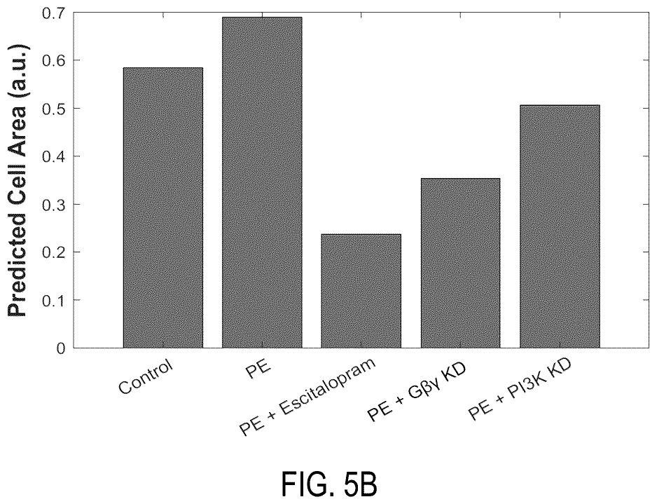

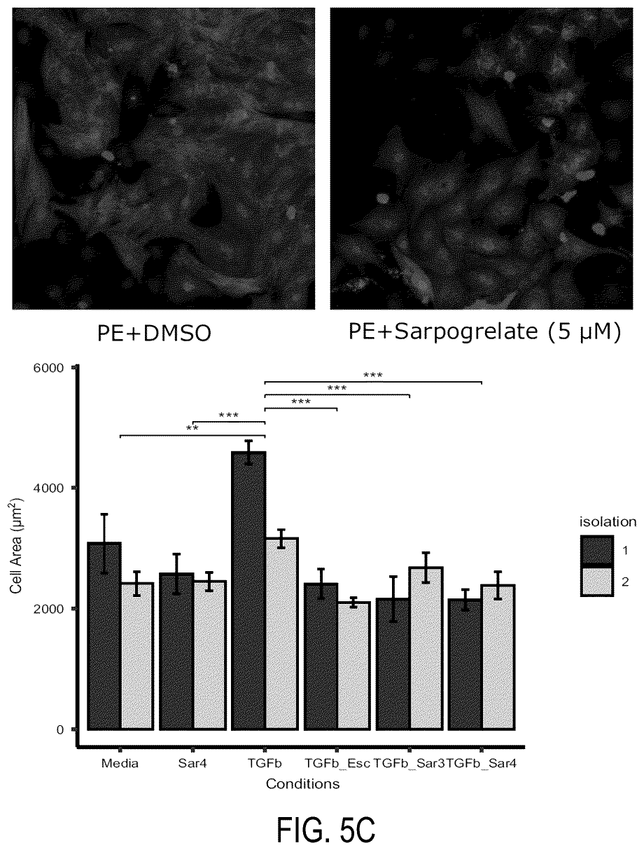

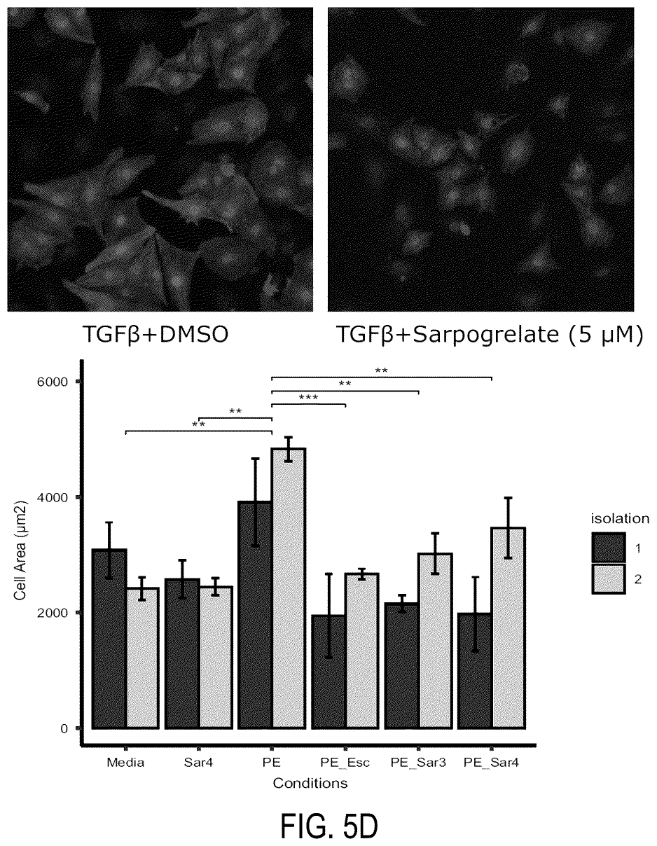

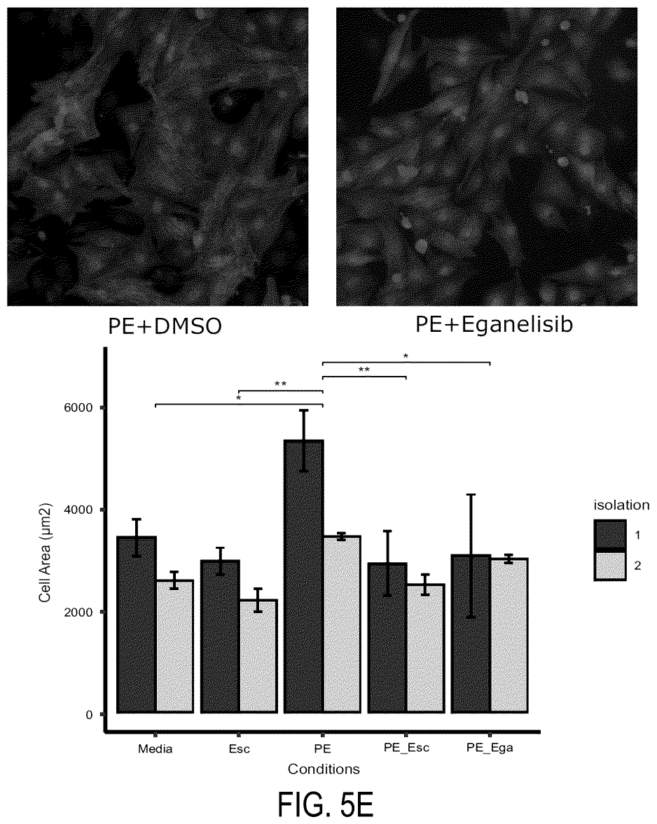

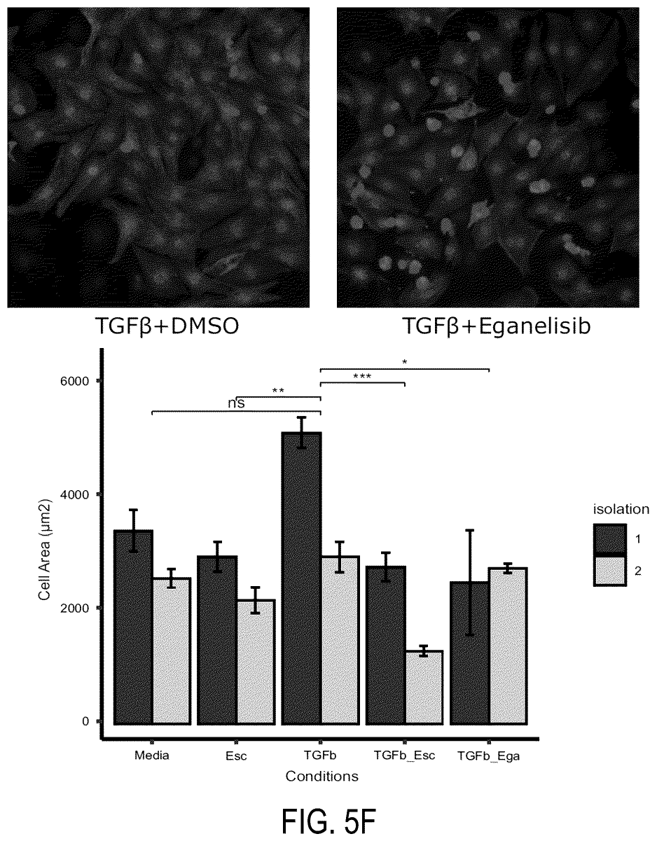

As the last stage of LogiRx, mechanistic subnetwork analysis was performed to identify pathways that mediate escitalopram inhibition of cardiomyocyte hypertrophy. The network-wide response to escitalopram was simulated, followed by simulations of network-wide knockdown of each network node in the presence and absence of escitalopram. The intersection of these two analyses resulted in a mechanistic subnetwork that predicted how escitalopram inhibits cardiomyocyte hypertrophy (FIG. 5A). This subnetwork predicted that escitalopram inhibits hypertrophy through the serotonin receptor HT2RA, a noncanonical target of escitalopram, rather than as a selective serotonin reuptake inhibitor (SSRI) via the transporter SERT (or SLC6A4). Further, the LogiRx model predicted that the escitalopram-HTR2A pathway acts by inhibiting G-protein signaling to PI3Kγ (FIGS. 5A and B). The model also correctly predicted how αMHC and βMHC gene expression respond to escitalopram.

To experimentally validate these LogiRx predictions, sarpogrelate hydrochloride was used which selectively inhibits HTR2A but not SERT. Prior literature demonstrates that sarpogrelate inhibits PE-induced cardiomyocyte hypertrophy both in vitro and in vivo. These studies confirm that sarpogrelate prevents both PE- and further demonstrates its efficacy against TGFβ-induced cardiomyocyte hypertrophy (FIGS. 5 C and D). Downstream of HTR2A, the mechanistic subnetwork predicted Gβγ and PI3K to mediate antihypertrophic escitalopram activity. Gβγ directly binds and activates PI3Kγ. To experimentally validate this prediction, PI3Kγ was selectively inhibited with the antitumor drug eganelisib. Eganelisib prevented both PE- and TGFβ-induced cardiomyocyte hypertrophy (FIGS. 5 E and F), further validating the noncanonical escitalopram-HTR2A-PI3Kγ pathway predicted by LogiRx.

### Escitalopram Inhibits Cardiomyocyte Hypertrophy In Vivo.

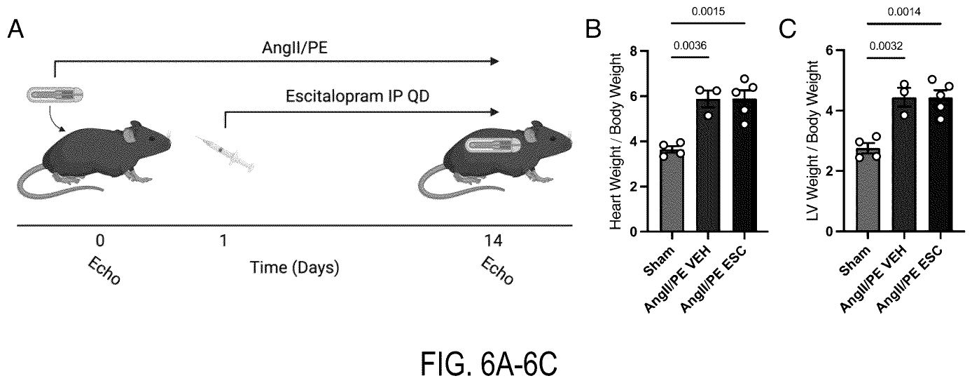

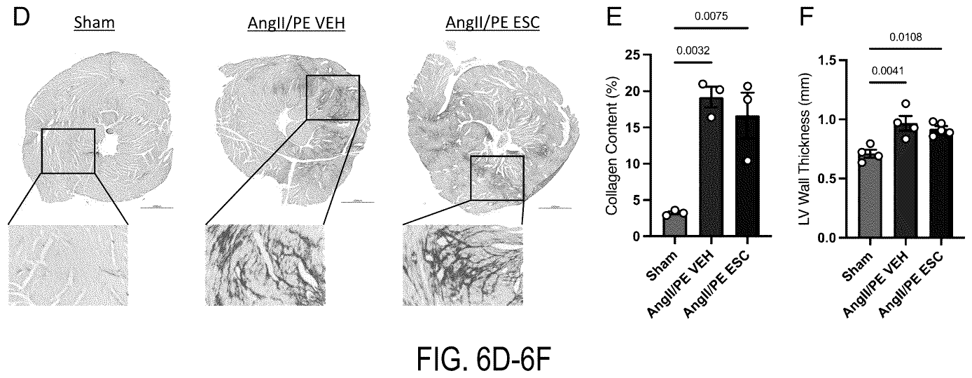

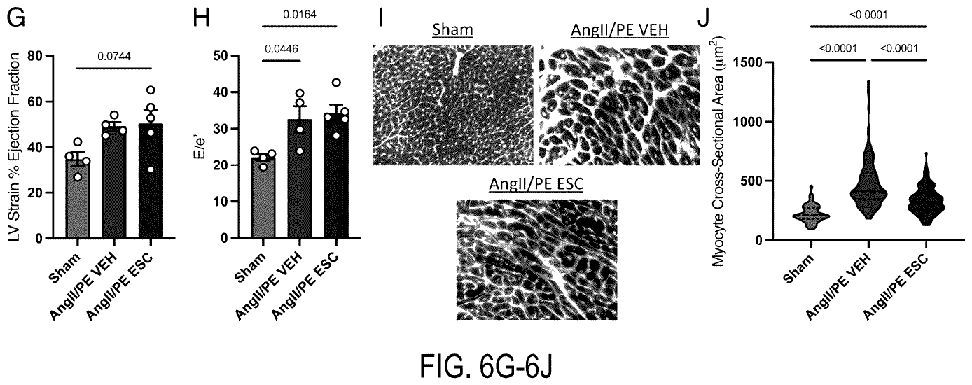

To test whether escitalopram also reduces cardiomyocyte hypertrophy in vivo, escitalopram was delivered to mice subjected to the angiotensin II and phenylephrine (AngII/PE) as a mouse model of cardiac hypertrophy and fibrosis. Angiotensin II (1.5 mg/kg/d) and phenylephrine (50 mg/kg/d) were delivered continuously for 14 d through an osmotic minipump as previously described; daily escitalopram was administered via intraperitoneal injection at 10 mg/kg/d beginning the day after pump implantation (FIG. 6A). Mice subjected to AngII/PE for 14 d exhibited significant global cardiac hypertrophy, as indicated by heart weight to body weight ratio (HW:BW), as well as a significant increase in LV mass in particular (LV:TL) (FIGS. 6 B and C). Furthermore, AngII/PE challenge induced dramatic fibrosis in the LV free wall, which likely also contributed to the increase in overall LV mass (FIGS. 6 D and E). Cardiac structure and systolic and diastolic function were evaluated by echocardiography, revealing an increase in LV wall thickness in diastole, which is consistent with the increased HW:BW ratio (FIG. 6F). Furthermore, AngII/PE mice demonstrated trends toward increases in systolic function, measured by ejection fraction, as well as increases in the E/E′ ratio, which is indicative of impaired LV relaxation or diastolic dysfunction (FIGS. 6 G and H).

Some mice treated with AngII/PE also received daily administration of escitalopram delivered via intraperitoneal injection at 10 mg/kg/d. These animals showed no change in overall collagen content in the LV, suggesting a lack of an effect on the cardiac fibroblast population (FIGS. 6 D and E). There was also no significant effect of escitalopram on global cardiac hypertrophy or any functional parameters assessed by echocardiography (FIGS. 6 B, C, and F-H). However, imaging of cardiomyocyte sections stained with WGA revealed a modest but significant reduction in individual cardiomyocyte hypertrophy in response to treatment with escitalopram (FIGS. 6 I and J). In summary, while escitalopram did not offer significant protection against global cardiac hypertrophy or dysfunction in this severe injury model, escitalopram attenuated cardiomyocyte hypertrophy in vivo, consistent with the antihypertrophic effect of escitalopram predicted by LogiRx and measured in cultured cardiomyocytes.

### Cardiac Outcomes for Patients Prescribed Escitalopram.

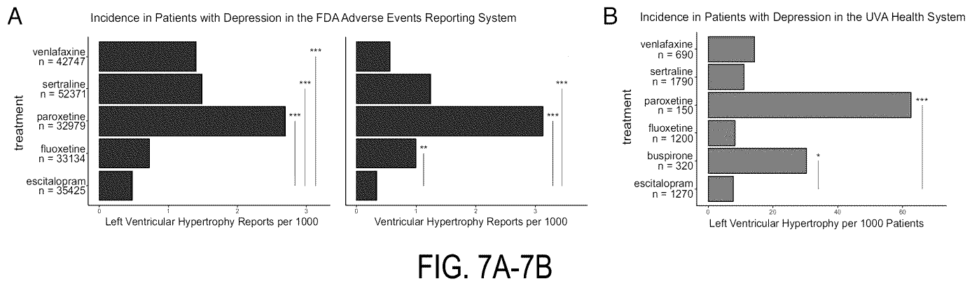

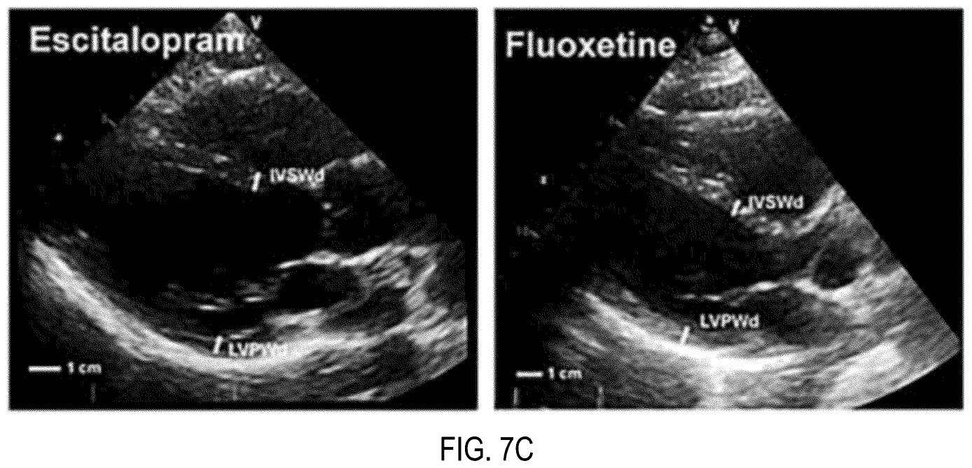

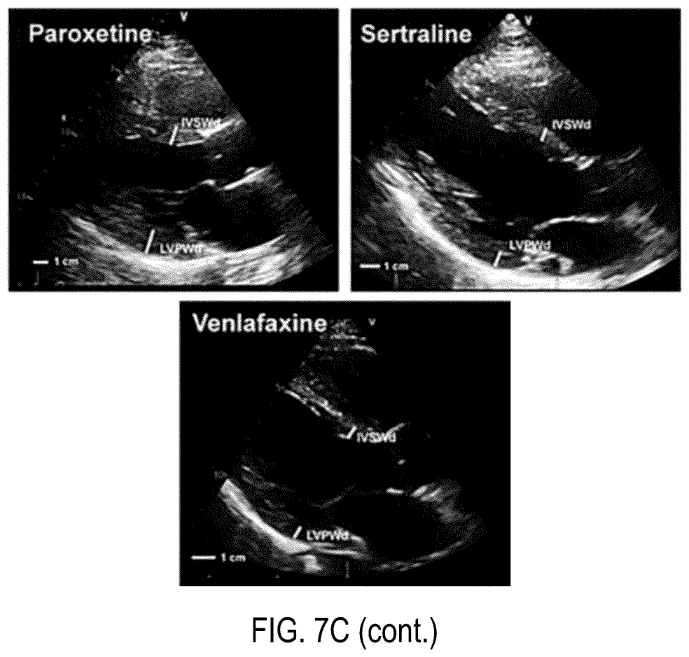

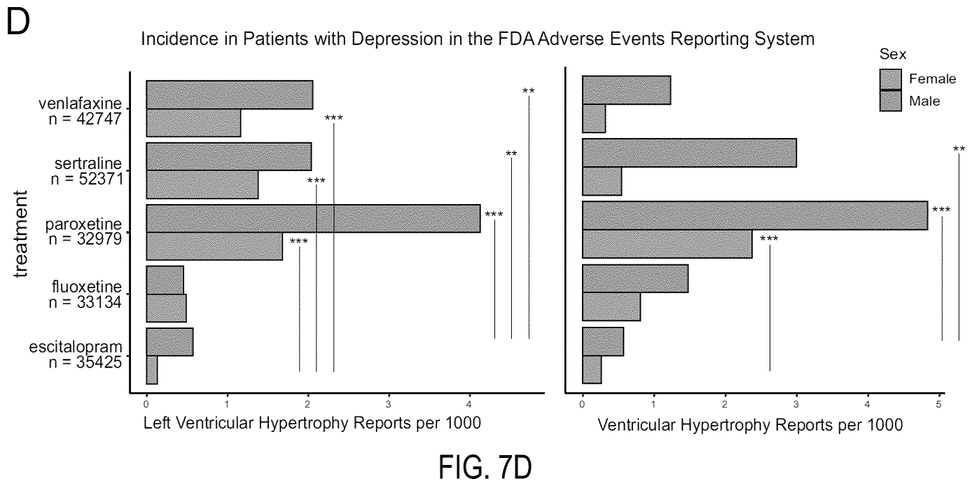

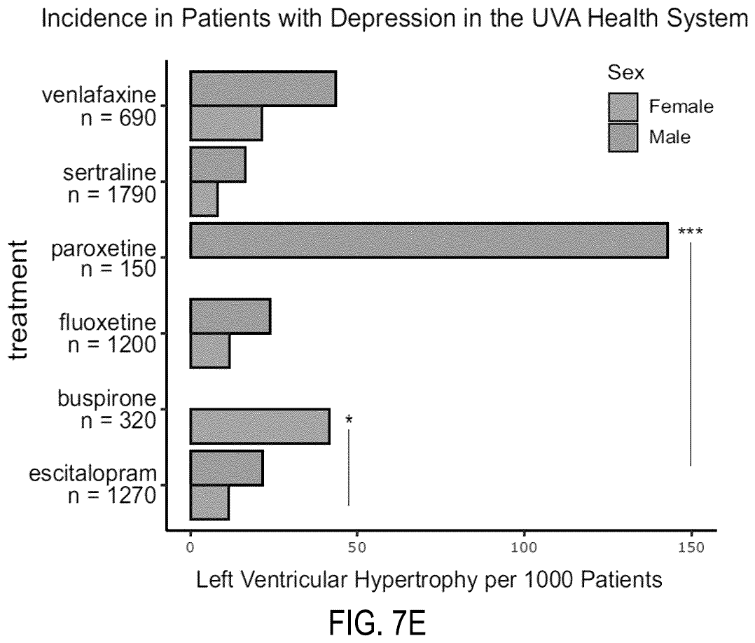

Based on consistent antihypertrophic roles of escitalopram shown by LogiRx, cultured rat cardiomyocytes, and mouse cardiomyocytes in vivo, whether patients prescribed escitalopram may also exhibit a reduced incidence of cardiac hypertrophy was investigated. Patient reports from the FDA Adverse Event Reporting System for incidence of cardiac hypertrophy or failure among patients prescribed escitalopram or related SSRIs that are not known to target HTR2A were first mined. Among those diagnosed with depression, patients prescribed escitalopram (which inhibits both SERT and HTR2A) had a lower incidence of left ventricular or ventricular hypertrophy when compared to treatment with other SSRIs sertraline, venlafaxine, and paroxetine that inhibit SERT but not HTR2A (FIG. 7A). An exception is patients treated with fluoxetine, who had lower incidence of cardiac failure compared to escitalopram treatment.

To validate these findings in an independent patient population with more structured data collection, electronic health records from the UVA Health System were mined. Among UVA patients diagnosed with depression, those prescribed escitalopram exhibited a statistically lower incidence of left ventricular hypertrophy than those prescribed SSRI paroxetine as well as the serotonin receptor agonist buspirone (FIG. 7B). To verify the hypertrophy annotation from this analysis, echocardiograms from deidentified patients using the same drug and diagnosis queries were examined. These confirm qualitatively larger interventricular septum thickness and left ventricular posterior wall end diastole in a patient prescribed paroxetine and diagnosed with hypertrophy than echocardiograms from patients prescribed escitalopram or other SSRIs not diagnosed with hypertrophy (FIG. 7C). Stratifying patients in the FDA (FIG. 7D) and UVA (FIG. 7E) databases by sex, both male and female patients with depression prescribed escitalopram exhibit less hypertrophy than that seen with two or more SSRIs. A reduced rate of hypertrophy was also seen in patients in the FDA database with anxiety on escitalopram compared with other SSRIs.

Methods of treating a patient having a given condition or at risk of the given condition may include administering to the patient a known, approved drug that was identified as affecting the given condition through use of the drug discovery systems described in the present disclosure. The treatment methods may utilize the computational predictions generated through the disclosed methods to identify therapeutic applications for existing pharmaceutical compounds that have established safety profiles and regulatory approval for other indications. The drug discovery system may enable identification of compounds with antihypertrophic activity through mechanistic pathway analysis and validation studies.

As one non-limiting example, the given condition may be cardiac hypertrophy, which may represent a pathological enlargement of cardiomyocytes that serves as a predictor of heart failure development. Cardiac hypertrophy may result from various physiological stressors, including pressure overload, drug toxicity, or genetic mutations that affect cardiac function. The treatment methods may target the underlying signaling pathways that regulate cardiomyocyte size and gene expression programs associated with hypertrophic remodeling.

Human patients suffering from cardiomyocyte hypertrophy or at risk of cardiomyocyte hypertrophy may be administered quantities of known, safe drugs identified through LogiRx analysis. The identified drugs may include OSI-930, crizotinib, escitalopram, tirbanibulin, rifabutin, mifepristone, and hesperidin, each of which may have been predicted to inhibit cardiomyocyte hypertrophy through distinct mechanistic pathways. These compounds may represent diverse therapeutic classes with different primary indications but may share the common property of modulating signaling networks that regulate cardiomyocyte growth.

OSI-930 may be administered to patients with cardiac hypertrophy based on its predicted activity as an FLT1 and KIT inhibitor that may modulate receptor tyrosine kinase signaling pathways involved in cardiomyocyte growth regulation. The compound may be originally designed for cancer treatment but may achieve antihypertrophic effects through inhibition of growth factor receptor cascades that contribute to cardiac remodeling. The dosing regimen for OSI-930 may be adapted from oncology applications while considering the different therapeutic context of cardiac hypertrophy treatment.

Crizotinib may be administered to patients with cardiac hypertrophy based on its predicted activity as an ALK and MET inhibitor that may affect protein kinase signaling networks regulating cardiomyocyte size and contractile protein expression. Although crizotinib may have known cardiac toxicity effects in cancer treatment, the compound may achieve beneficial antihypertrophic effects through specific pathway interactions that differ from its toxicity mechanisms. The therapeutic application may require careful monitoring and dose optimization to maximize antihypertrophic benefits while minimizing adverse cardiac effects.

Escitalopram may be administered to patients with cardiac hypertrophy based on its predicted off-target activity through HTR2A serotonin receptor antagonism and downstream PI3Kγ pathway inhibition. The compound may be commonly prescribed for depression and anxiety disorders with an established safety profile, making it a candidate for drug repurposing applications in cardiac conditions. The antihypertrophic effects of escitalopram may occur independently of its primary serotonin reuptake inhibition mechanism, enabling therapeutic benefits in patients without psychiatric indications.

The dosing regimen for escitalopram in cardiac hypertrophy treatment may be based on the established dosing ranges used for psychiatric indications, with potential modifications based on the different therapeutic target and patient population. The compound may be administered orally at doses ranging from 5 mg to 20 mg daily, with dose titration based on patient response and tolerance. The treatment duration may extend over months or years depending on the progression of cardiac hypertrophy and the patient's overall cardiovascular risk profile.

Tirbanibulin may be administered to patients with cardiac hypertrophy based on its predicted activity as a TUBB and SRC inhibitor that may modulate cytoskeletal organization and kinase signaling pathways regulating cardiomyocyte structure and function. The compound may be approved for actinic keratosis treatment and may be under investigation for acute myeloid leukemia, indicating established safety data that may support cardiac applications. The mechanism of action through cytoskeletal protein targeting may provide a novel approach to cardiac hypertrophy treatment that differs from conventional cardiovascular therapies.

Rifabutin may be administered to patients with cardiac hypertrophy based on its predicted activity as an HSP90AA1 inhibitor that may affect molecular chaperone networks regulating protein stability and cellular stress responses in cardiomyocytes. The compound may be approved as an antibiotic for Mycobacterium avium complex prevention in HIV patients, providing established safety and dosing information that may be adapted for cardiac applications. The heat shock protein targeting mechanism may address the protein folding stress that contributes to hypertrophic cardiomyocyte remodeling.

Mifepristone may be administered to patients with cardiac hypertrophy based on its predicted activity through glucocorticoid receptor antagonism and downstream LCK/ERK and CEBPβ pathway modulation. The compound may be approved for use as an abortifacient and for treating Cushing's syndrome with hyperglycemia, indicating established clinical experience with steroid hormone receptor antagonism. The glucocorticoid receptor targeting mechanism may provide therapeutic benefits by interrupting hormone-mediated signaling pathways that contribute to cardiomyocyte hypertrophy.

The dosing regimen for mifepristone in cardiac hypertrophy treatment may be adapted from its approved indications while considering the chronic nature of cardiac remodeling processes. The compound may be administered orally at doses ranging from 200 mg to 600 mg daily, with careful monitoring for potential side effects related to glucocorticoid receptor antagonism. The treatment approach may require coordination with endocrinology specialists to manage potential effects on cortisol signaling and adrenal function.

Hesperidin may be administered to patients with cardiac hypertrophy based on its predicted activity as a flavonoid that may modulate inflammatory signaling pathways and oxidative stress responses contributing to cardiomyocyte remodeling. The compound may be available as a natural supplement with anti-inflammatory properties, providing a potentially safer option for long-term cardiac hypertrophy management. The antioxidant and anti-inflammatory mechanisms may address multiple pathological processes involved in cardiac remodeling beyond direct hypertrophic signaling pathways.

The treatment methods may involve combination therapy approaches that utilize multiple drugs identified through LogiRx analysis to target different aspects of the hypertrophic signaling network. Combination therapy may provide enhanced therapeutic efficacy by addressing multiple pathways simultaneously while potentially reducing the required dose of individual compounds. The mechanistic diversity of the identified drugs may enable rational combination design based on complementary pathway targeting and synergistic effects.

Patient selection for treatment with drugs identified through LogiRx analysis may involve assessment of cardiac hypertrophy severity, underlying etiology, and individual patient factors that may influence drug response. Patients with early-stage hypertrophy may be candidates for preventive treatment approaches, while patients with established hypertrophy may require more intensive therapeutic interventions. The mechanistic predictions from LogiRx analysis may guide selection of appropriate drugs based on the specific pathways involved in individual patient presentations.

Monitoring of treatment response may involve serial echocardiographic assessment of cardiac dimensions and function, measurement of hypertrophic biomarkers, and evaluation of clinical symptoms related to cardiac performance. The treatment response monitoring may enable dose optimization and assessment of therapeutic efficacy over time. The mechanistic understanding provided by LogiRx analysis may guide selection of appropriate biomarkers and monitoring parameters that reflect the specific pathways targeted by each therapeutic compound.

The treatment methods may be particularly applicable to patients with cardiac hypertrophy secondary to hypertension, valvular disease, or genetic cardiomyopathies where conventional therapies may provide incomplete benefit. The novel mechanisms identified through LogiRx analysis may offer therapeutic options for patients who do not respond adequately to standard cardiovascular medications such as ACE inhibitors, beta blockers, or angiotensin receptor blockers. The drug repurposing approach may provide access to established compounds with known safety profiles while addressing unmet therapeutic needs in cardiac hypertrophy management.

Safety monitoring for treatment with repurposed drugs may involve assessment of both cardiovascular effects and potential side effects related to the compounds' primary therapeutic indications. Patients receiving escitalopram for cardiac hypertrophy may require monitoring for psychiatric effects, while patients receiving mifepristone may require monitoring for endocrine effects. The established safety profiles of these compounds from their approved indications may provide guidance for risk assessment and monitoring protocols in cardiac applications.

The treatment duration and long-term management strategies may depend on the underlying cause of cardiac hypertrophy and the patient's response to therapy. Patients with reversible causes of hypertrophy may require shorter treatment courses, while patients with progressive conditions may require long-term therapy to prevent further cardiac remodeling. The mechanistic insights from LogiRx analysis may inform decisions about treatment duration by identifying whether the targeted pathways require continuous inhibition or whether intermittent therapy may be sufficient to maintain therapeutic benefits.

The systems and methods described in the present disclosure may be applied to different cellular phenotypes beyond cardiomyocyte hypertrophy through adaptation of the computational framework to alternative signaling network models and cellular response systems. The method may be amenable for integrating drug screens and mechanistic network models in various biological areas where annotated drugs have been used to screen cellular phenotype or gene expression and where validated mechanistic network models exist for the cellular processes of interest.

Myofibroblast transformation for fibrosis may represent one application area where the LogiRx method may provide mechanistic insights into drug effects on cellular differentiation processes. Myofibroblast transformation may involve the conversion of quiescent fibroblasts into activated myofibroblasts that produce excessive extracellular matrix proteins and contribute to tissue fibrosis. The transformation process may be regulated by complex signaling networks that include TGF-β signaling, mechanical stress pathways, and inflammatory mediator cascades.

The application of LogiRx to myofibroblast transformation may utilize validated mechanistic network models that capture the signaling pathways regulating fibroblast activation and differentiation. These network models may include nodes representing key regulatory proteins such as TGF-β receptors, Smad transcription factors, α-smooth muscle actin, and collagen synthesis enzymes. The edges in the network may represent regulatory interactions that control the transition from quiescent fibroblasts to activated myofibroblasts.

Drug screening data for compounds that modulate myofibroblast transformation may be integrated with the fibrosis network model through the LogiRx pathway mapping approach. Compounds that inhibit or promote myofibroblast differentiation may be mapped to their protein targets using drug-target databases, and potential pathways connecting these targets to the fibrosis network may be identified through protein interaction database searches. The pathway optimization algorithms may rank potential mechanistic routes by which drugs influence myofibroblast transformation.

The logic-based simulation component of LogiRx may enable prediction of drug effects on myofibroblast marker expression, extracellular matrix production, and contractile properties that characterize the activated myofibroblast phenotype. The simulations may account for different fibrotic stimuli and may predict context-dependent drug efficacy across various tissue types and disease models. The mechanistic subnetwork analysis may identify specific pathway components that mediate drug effects on myofibroblast transformation.

Proliferation for breast cancer may represent another application area where LogiRx may provide mechanistic understanding of drug effects on cellular growth and division processes. Breast cancer cell proliferation may be regulated by complex signaling networks that include growth factor receptor pathways, cell cycle control mechanisms, apoptosis regulation, and metabolic reprogramming cascades. The proliferation phenotype may involve changes in DNA synthesis, mitotic progression, and cell survival that can be measured through various experimental approaches.

The application of LogiRx to breast cancer proliferation may utilize validated mechanistic network models that capture the signaling pathways regulating cancer cell growth and survival. These network models may include nodes representing oncogenes, tumor suppressor proteins, cell cycle regulators, and apoptosis mediators that control cancer cell proliferation. The network structure may reflect the dysregulated signaling patterns characteristic of breast cancer cells compared to normal mammary epithelial cells.

Drug screening data for compounds that affect breast cancer cell proliferation may be integrated with the cancer network model through LogiRx pathway mapping methods. Anti-proliferative compounds identified through high-throughput screening may be mapped to their molecular targets, and pathways connecting these targets to proliferation control networks may be identified through systematic pathway search algorithms. The pathway ranking system may prioritize mechanistic routes that are most likely to mediate anti-proliferative drug effects.

The logic-based simulation framework may enable prediction of drug effects on cell cycle progression, apoptosis induction, and growth factor signaling that regulate breast cancer cell proliferation. The simulations may account for different cancer subtypes and may predict drug efficacy across various genetic backgrounds and treatment contexts. The mechanistic subnetwork analysis may reveal specific pathway components that are required for drug-mediated growth inhibition.

The performance of LogiRx in alternative cellular phenotype applications may depend on the accuracy and completeness of the underlying network models and the quality of drug screening data available for the specific cellular processes. Network models with extensive experimental validation and comprehensive pathway coverage may provide more reliable predictions than models with limited validation or incomplete pathway representation. The availability of high-quality drug-target annotation and protein interaction data may also influence the success of pathway mapping efforts.

The characteristics of the validation experiments used to assess network model accuracy may affect the reliability of LogiRx predictions for different cellular phenotypes. Network models validated against diverse experimental conditions and multiple cellular readouts may provide more robust predictions than models validated against limited experimental datasets. The validation experiments may need to encompass the range of cellular responses and drug effects that are relevant to the specific application area.

The LogiRx method may be most readily applied to cellular phenotypes where comprehensive drug screening has been performed using annotated compound libraries with known target information. The availability of screening data that links specific compounds to phenotypic effects may enable systematic analysis of drug mechanisms through pathway mapping and network simulation approaches. The compound annotation quality may directly impact the ability to identify reliable pathway connections between drug targets and cellular phenotype networks.

The integration of LogiRx with different types of cellular phenotype measurements may require adaptation of the simulation and analysis methods to account for the specific readouts used in each application area. Proliferation assays may measure different cellular parameters than differentiation assays, requiring corresponding modifications to the network model outputs and validation criteria. The mechanistic subnetwork analysis may need to be tailored to identify pathway components that are most relevant to the specific phenotypic measurements used in each application.

The scalability of the LogiRx approach may enable application to large-scale drug discovery programs that screen extensive compound libraries against multiple cellular phenotypes simultaneously. The computational framework may handle parallel analysis of different network models and may identify compounds with multi-target effects across different cellular processes. This capability may support identification of compounds with broad therapeutic potential or may reveal unexpected connections between different disease mechanisms.

The drug repurposing applications of LogiRx may be particularly valuable for cellular phenotypes where existing therapeutic options are limited or where novel mechanisms of action may provide therapeutic advantages. The method may identify approved drugs with previously unrecognized effects on specific cellular processes, potentially accelerating the development of new therapeutic applications. The mechanistic insights provided by LogiRx may support regulatory approval processes by providing scientific rationale for drug repurposing applications.

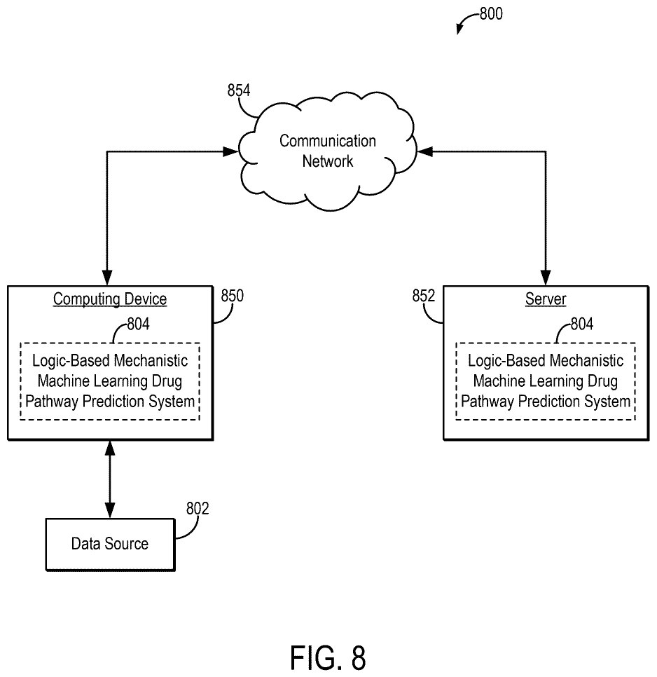

FIG. 8 shows an example of a system 800 for logic-based machine learning prediction of drug pathways that modulate cellular phenotypes in accordance with some embodiments described in the present disclosure. As shown in FIG. 8, a computing device 850 can receive one or more types of data (e.g., signaling network model data, drug-target database data, protein interaction database data) from data source 802. In some embodiments, computing device 850 can execute at least a portion of a logic-based mechanistic machine learning drug pathway prediction system 804 to predict drug pathways and their effects on cellular phenotypes from data received from the data source 802.

Additionally or alternatively, in some embodiments, the computing device 850 can communicate information about data received from the data source 802 to a server 852 over a communication network 854, which can execute at least a portion of the logic-based mechanistic machine learning drug pathway prediction system 804. In such embodiments, the server 852 can return information to the computing device 850 (and/or any other suitable computing device) indicative of an output of the logic-based mechanistic machine learning drug pathway prediction system 804.

In some embodiments, computing device 850 and/or server 852 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 802 can be any suitable source of data (e.g., signaling network model data, drug-target database data, protein interaction database data), another computing device (e.g., a server storing signaling network model data, drug-target database data, protein interaction database data), and so on. In some embodiments, data source 802 can be local to computing device 850. For example, data source 802 can be incorporated with computing device 850 (e.g., computing device 850 can be configured as part of a device for measuring, recording, estimating, acquiring, or otherwise collecting or storing data). As another example, data source 802 can be connected to computing device 850 by a cable, a direct wireless link, and so on. Additionally or alternatively, in some embodiments, data source 802 can be located locally and/or remotely from computing device 850, and can communicate data to computing device 850 (and/or server 852) via a communication network (e.g., communication network 854).

In some embodiments, communication network 854 can be any suitable communication network or combination of communication networks. For example, communication network 854 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 854 can be a local area network, a wide area network, a public network (e.g., the Internet), a private or semi-private network (e.g., a corporate or university intranet), any other suitable type of network, or any suitable combination of networks. Communications links shown in FIG. 8 can each be any suitable communications link or combination of communications links, such as wired links, fiber optic links, Wi-Fi links, Bluetooth links, cellular links, and so on.

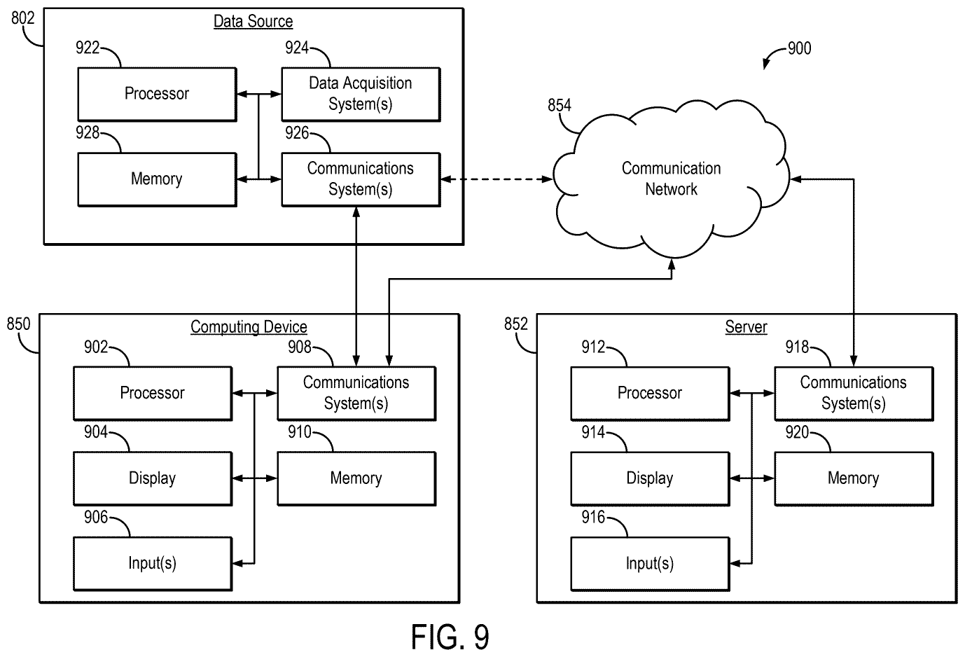

Referring now to FIG. 9, an example of hardware 900 that can be used to implement data source 802, computing device 850, and server 852 in accordance with some embodiments of the systems and methods described in the present disclosure is shown.

As shown in FIG. 9, in some embodiments, computing device 850 can include a processor 902, a display 904, one or more inputs 906, one or more communication systems 908, and/or memory 910. In some embodiments, processor 902 can be any suitable hardware processor or combination of processors, such as a central processing unit (CPU), a graphics processing unit (GPU), and so on. In some embodiments, display 904 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 906 can include any suitable input devices and/or sensors that can be used to receive user input, such as a keyboard, a mouse, a touchscreen, a microphone, and so on.

In some embodiments, communications systems 908 can include any suitable hardware, firmware, and/or software for communicating information over communication network 854 and/or any other suitable communication networks. For example, communications systems 908 can include one or more transceivers, one or more communication chips and/or chip sets, and so on. In a more particular example, communications systems 908 can include hardware, firmware, and/or software that can be used to establish a Wi-Fi connection, a Bluetooth connection, a cellular connection, an Ethernet connection, and so on.

In some embodiments, memory 910 can include any suitable storage device or devices that can be used to store instructions, values, data, or the like, that can be used, for example, by processor 902 to present content using display 904, to communicate with server 852 via communications system(s) 908, and so on. Memory 910 can include any suitable volatile memory, non-volatile memory, storage, or any suitable combination thereof. For example, memory 910 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 910 can have encoded thereon, or otherwise stored therein, a computer program for controlling operation of computing device 850. In such embodiments, processor 902 can execute at least a portion of the computer program to present content (e.g., images, user interfaces, graphics, tables), receive content from server 852, transmit information to server 852, and so on. For example, the processor 902 and the memory 910 can be configured to perform the methods described herein (e.g., the method of FIG. 1).

In some embodiments, server 852 can include a processor 912, a display 914, one or more inputs 916, one or more communications systems 918, and/or memory 920. In some embodiments, processor 912 can be any suitable hardware processor or combination of processors, such as a CPU, a GPU, and so on. In some embodiments, display 914 can include any suitable display devices, such as an LCD screen, LED display, OLED display, electrophoretic display, a computer monitor, a touchscreen, a television, and so on. In some embodiments, inputs 916 can include any suitable input devices and/or sensors that can be used to receive user input, such as a keyboard, a mouse, a touchscreen, a microphone, and so on.

In some embodiments, communications systems 918 can include any suitable hardware, firmware, and/or software for communicating information over communication network 854 and/or any other suitable communication networks. For example, communications systems 918 can include one or more transceivers, one or more communication chips and/or chip sets, and so on. In a more particular example, communications systems 918 can include hardware, firmware, and/or software that can be used to establish a Wi-Fi connection, a Bluetooth connection, a cellular connection, an Ethernet connection, and so on.

In some embodiments, memory 920 can include any suitable storage device or devices that can be used to store instructions, values, data, or the like, that can be used, for example, by processor 912 to present content using display 914, to communicate with one or more computing devices 850, and so on. Memory 920 can include any suitable volatile memory, non-volatile memory, storage, or any suitable combination thereof. For example, memory 920 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 920 can have encoded thereon a server program for controlling operation of server 852. In such embodiments, processor 912 can execute at least a portion of the server program to transmit information and/or content (e.g., data, images, a user interface) to one or more computing devices 850, receive information and/or content from one or more computing devices 850, receive instructions from one or more devices (e.g., a personal computer, a laptop computer, a tablet computer, a smartphone), and so on.

In some embodiments, the server 852 is configured to perform the methods described in the present disclosure. For example, the processor 912 and memory 920 can be configured to perform the methods described herein (e.g., the method of FIG. 1).

In some embodiments, data source 802 can include a processor 922, one or more inputs 924, one or more communications systems 926, and/or memory 928. In some embodiments, processor 922 can be any suitable hardware processor or combination of processors, such as a CPU, a GPU, and so on. In some embodiments, the one or more inputs 924 are generally configured to acquire data and can include a database interface for accessing drug-target databases such as DrugBank, a network interface for retrieving protein interaction data from databases such as OmniPath, a file input system for loading signaling network model data, or an API interface for accessing experimental validation datasets. Additionally or alternatively, in some embodiments, the one or more inputs 924 can include any suitable hardware, firmware, and/or software for coupling to and/or controlling operations of a high-throughput screening system, microscopy equipment for cellular imaging, or laboratory information management systems. In some embodiments, one or more portions of the input(s) 924 can be removable and/or replaceable.

Note that, although not shown, data source 802 can include any suitable inputs and/or outputs. For example, data source 802 can include input devices and/or sensors that can be used to receive user input, such as a keyboard, a mouse, a touchscreen, a microphone, a trackpad, a trackball, and so on. As another example, data source 802 can include any suitable display devices, such as an LCD screen, an LED display, an OLED display, an electrophoretic display, a computer monitor, a touchscreen, a television, etc., one or more speakers, and so on.

In some embodiments, communications systems 926 can include any suitable hardware, firmware, and/or software for communicating information to computing device 850 (and, in some embodiments, over communication network 854 and/or any other suitable communication networks). For example, communications systems 926 can include one or more transceivers, one or more communication chips and/or chip sets, and so on. In a more particular example, communications systems 926 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 928 can include any suitable storage device or devices that can be used to store instructions, values, data, or the like, that can be used, for example, by processor 922 to control the one or more inputs 924, and/or receive data from the one or more inputs 924; to generate images from data; present content (e.g., data, images, a user interface) using a display; communicate with one or more computing devices 850; and so on. Memory 928 can include any suitable volatile memory, non-volatile memory, storage, or any suitable combination thereof. For example, memory 928 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 928 can have encoded thereon, or otherwise stored therein, a program for controlling operation of data source 802. In such embodiments, processor 922 can execute at least a portion of the program to generate images, transmit information and/or content (e.g., data, images, a user interface) to one or more computing devices 850, receive information and/or content from one or more computing devices 850, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

In some embodiments, the logic-based mechanistic machine learning drug pathway prediction system 804 may be configured as a drug discovery system. In these instances, the drug discovery system may include various hardware and software components configured to perform logic-based machine learning prediction of drug pathways. The system 804 may include a processor and a memory having stored thereon a set of instructions which, when executed by the processor, cause the processor to perform various computational operations for drug pathway analysis and prediction.

The system 804 may further comprise a first memory storage and a second memory storage that contain databases for drug discovery applications. The first memory storage may include a first database containing drug-target information, while the second memory storage may include a second database containing protein interaction data. These memory storage components may be located on the same computing device or server (such as computing device 850 or server 852) or may be distributed amongst multiple computing devices, servers, or other hardware components within the system 804.

The processor of system 804 may be configured to execute the set of instructions stored in the memory to perform multiple computational operations. The processor may obtain a logic-based model of a signaling network relating to a given condition, where the logic-based model includes a plurality of nodes and a plurality of edges representing molecular interactions and regulatory relationships. The processor may access this model from the memory components of system 804 or from external data sources through network connections via communication network 854.

The set of instructions within system 804 may cause the processor to determine a set of candidate compounds that may affect the given condition. This determination may involve analyzing screening data, literature information, or other sources of compound activity data received from data source 802. The processor may filter and prioritize compounds based on various criteria, such as known biological activity, safety profiles, or availability for testing.

The processor of system 804 may identify protein targets for the candidate compounds by accessing drug-target databases stored in the first memory storage. The identification process may involve querying the first database with compound identifiers and retrieving associated protein target information. The processor may handle multiple target assignments for individual compounds and may filter targets based on confidence scores or annotation quality.

The set of instructions within system 804 may further cause the processor to determine potential connections from the candidate compounds to the given condition via the logic-based model. This determination may involve computing a ranked list of pathways through searching a directed protein interaction network and minimizing a cost function of the product of edge weights along each pathway. The processor may access protein interaction data from the second database stored in the second memory storage, which may be located on the same hardware component as the first memory storage or distributed across different computing devices or servers within system 804.

The processor of system 804 may implement pathway optimization algorithms that search through the directed protein interaction network to identify routes connecting drug targets to nodes within the logic-based signaling network model. The algorithms may evaluate multiple potential pathways and rank them according to cost functions that consider factors such as pathway length, interaction confidence scores, and network topology characteristics.

The set of instructions within system 804 may cause the processor to output the ranked list of pathways to a user for further analysis and drug discovery applications. The output may be provided through various interfaces associated with computing device 850 or server 852, such as graphical displays, data files, or network visualizations. The processor may format the output to include pathway information, confidence scores, and mechanistic predictions that support drug discovery decision-making processes.

The memory components of system 804 may store additional instructions that cause the processor to perform mechanistic subnetwork analysis to explain drug response mechanisms. This analysis may involve simulating network responses to drug perturbations and identifying specific network components that mediate drug effects. The processor may systematically test the contribution of individual network nodes to observed drug responses and determine mechanistic subnetworks that explain drug action.

The system 804 may be configured to handle multiple types of conditions and cellular phenotypes. The processor may access different logic-based models corresponding to various biological processes stored across the memory components and may adapt the pathway search algorithms accordingly. The system architecture may support scalable analysis of large compound libraries and may integrate with external databases and computational resources through communication network 854 as needed for comprehensive drug discovery applications.

As described above, the first memory storage may include a drug-target database that contains information about molecular targets for pharmaceutical compounds. In some cases, the drug-target database stored in the first memory storage may be DrugBank, which provides comprehensive data linking drugs to their known protein targets. The first memory storage may store target identification information that enables mapping of candidate compounds to specific proteins within cellular signaling networks, and may be located on computing device 850, server 852, or data source 802.

The system 804 may utilize the drug-target database stored in the first memory storage for identifying protein targets of candidate compounds. The processor 902 or 912 may execute instructions to identify protein targets by accessing the drug-target database to query compound identifiers and retrieve associated protein target information. In some cases, the DrugBank database stored in memory 910 or 920 may provide target annotations that include binding affinities, interaction mechanisms, and confidence scores for drug-protein relationships.

The second memory storage may include a protein interaction database that contains data about how proteins interact within cellular networks. In some cases, the protein interaction database stored in the second memory storage may be the Omnipath database, which aggregates protein interaction information from multiple literature sources. The second memory storage may store directed interaction data that describes regulatory relationships between proteins in signaling pathways, and may be located on the same hardware component as the first memory storage or distributed across different computing devices or servers within system 804.

The system 804 may utilize the protein interaction database stored in the second memory storage for determining potential connections between drug targets and cellular phenotypes. The processor 902 or 912 may execute instructions to determine potential connections by accessing the protein interaction database to search for pathways that link drug targets to nodes within logic-based signaling network models. The Omnipath database stored in memory 910 or 920 may provide directed interaction data that enables pathway optimization algorithms to identify routes through protein interaction networks.

The process of drug target identification executed by processor 902 or 912 may involve filtering drug targets based on annotations present in the protein interaction database stored in the second memory storage. Drug targets identified from the drug-target database in the first memory storage may be cross-referenced with protein annotations in the protein interaction database to ensure compatibility with pathway mapping algorithms. This filtering process executed by the processor may remove drug targets that lack sufficient interaction data or that are not well-connected to the signaling network of interest.

In some cases, the system 804 may successfully map a subset of candidate compounds to protein targets through the combined use of both the first and second memory storage databases. For example, analysis of antihypertrophic compounds executed by processor 902 or 912 may result in successful mapping of compounds to protein targets using the drug-target database and protein interaction database filtering process. The mapped compounds may represent those with sufficient target annotation and protein interaction data stored in memory 910 or 920 to enable pathway analysis.

The pathway mapping process executed by processor 902 or 912 may further connect the identified drug targets to signaling network models through protein interaction pathways stored in the second memory storage. In some cases, a subset of the successfully mapped compounds may be connected to a hypertrophy network via candidate drug pathways identified by the system 804. These pathways may represent potential routes through which the compounds may influence cellular phenotypes related to the given condition.

The integration of drug-target and protein interaction databases stored in the first and second memory storage components of system 804 may enable comprehensive analysis of compound mechanisms. The first memory storage may provide the initial mapping between compounds and their direct molecular targets, while the second memory storage may provide the network context that enables prediction of downstream effects. This dual-database approach implemented by processor 902 or 912 may support both direct target identification and indirect pathway analysis for drug discovery applications.

The filtering process using protein interaction database annotations executed by processor 902 or 912 may improve the quality of pathway predictions by focusing on well-characterized protein interactions stored in memory 910 or 920. Compounds that map to protein targets with extensive interaction data may be more likely to yield reliable pathway predictions than compounds targeting poorly characterized proteins. The annotation filtering step executed by the processor may therefore serve as a quality control mechanism that enhances the reliability of subsequent pathway analysis steps performed by system 804.

The drug discovery system 800 may operate through an integrated workflow that combines multiple computational components to predict mechanistic pathways by which drugs affect cellular phenotypes. The system 804 may obtain a logic-based model of a signaling network relating to a given condition from data source 802, where the model includes a plurality of nodes representing proteins or mRNAs and a plurality of edges representing regulatory interactions between these molecular components.

In some cases, the system 804 may determine a set of candidate compounds that may affect the given condition through analysis of experimental screening data or literature-based compound libraries received from data source 802. The processor 902 or 912 may identify protein targets for these candidate compounds by querying the first database stored in memory 910 or 920, which may contain drug-target interaction data. The system 804 may filter these protein targets based on their presence in the second database containing protein interaction network information stored in memory 910 or 920.

The pathway identification process executed by processor 902 or 912 may involve computing a ranked list of pathways by searching the directed protein interaction network and minimizing a cost function based on the product of edge weights along each pathway. The system 804 may employ path optimization algorithms, such as PathLinker, to identify top-scoring directed pathways from drug targets to nodes within the signaling network model. In some embodiments, the cost function may incorporate edge weights that reflect the confidence or strength of protein-protein interactions stored in memory 910 or 920.

The system 804 may create expanded logic-based differential equation model variants by individually adding each identified drug pathway to the base signaling network model stored in memory 910 or 920. Each model variant may simulate the functional impact of incorporating a specific drug pathway on the cellular phenotype of interest. The processor 902 or 912 may test these expanded models by simulating inhibition or activation of drug targets in the presence of relevant stimuli.

In some cases, the system 804 may validate the functional validity of identified drug pathways by testing their performance against experimental literature data stored in memory 910 or 920. The system 804 may maintain model accuracy above a predetermined threshold when incorporating new drug pathways. Drug pathways that reduce model accuracy below this threshold may be excluded from further analysis by processor 902 or 912.

The mechanistic subnetwork analysis component of system 804 may identify specific pathways that mediate drug response by simulating the global response of the network to drug treatment in the presence of relevant stimuli. The system 804 may identify drug-responsive nodes by comparing network states with and without drug treatment using processor 902 or 912. The processor 902 or 912 may then simulate knockdown of each node and measure that node's influence on drug-induced changes in the cellular phenotype, thereby identifying regulating nodes.

The system 804 may determine mechanistic subnetworks by computing the intersection of drug-responsive nodes with regulating nodes using processor 902 or 912. These mechanistic subnetworks may map the pathways that mediate drug activity and provide mechanistic understanding of how drugs modulate cellular processes. In some embodiments, the mechanistic subnetworks may reveal off-target pathways through which drugs affect cellular phenotypes, enabling drug repurposing applications supported by system 804.

The integrated workflow of system 804 may combine drug-target databases, protein interaction databases, path optimization algorithms, and logic-based network simulations to provide both predictive capabilities and mechanistic insights. The system 804 may output ranked lists of drug pathways along with associated mechanistic subnetworks to users through computing device 850 or server 852 for further analysis and drug discovery applications. In some cases, the output may include predictions of drug efficacy across multiple biochemical environments or cellular contexts generated by processor 902 or 912.

The system 804 may enable drug repurposing by revealing how existing drugs may modulate cellular processes through pathways that differ from their primary therapeutic mechanisms. The processor 902 or 912 may identify drugs that target the signaling network through indirect pathways involving intermediate proteins not present in the original network model stored in memory 910 or 920. In some embodiments, the system 804 may predict context-dependent drug efficacy by simulating drug effects across different hypertrophic stimuli or cellular conditions.

The integrated system 804 may support validation of drug predictions through multiple approaches, including experimental testing in cellular models, animal studies, and analysis of patient databases accessed through communication network 854. The system 804 may provide mechanistic hypotheses that guide experimental design and interpretation of validation studies. In some cases, the system 804 may generate predictions that can be tested using specific pharmacological tools or genetic perturbations to validate predicted pathways.

The present disclosure has described one or more preferred embodiments, and it should be appreciated that many equivalents, alternatives, variations, and modifications, aside from those expressly stated, are possible and within the scope of the disclosure.

1. A computer-implemented method for predicting drug pathways that modulate cellular phenotypes, comprising:
obtaining a logic-based differential equation model of a signaling network relating to a cellular phenotype, the model comprising a plurality of nodes representing proteins or mRNAs and a plurality of edges representing interactions between the nodes;
identifying a set of candidate compounds that affect the cellular phenotype;
determining protein targets for each candidate compound using a drug-target database;
mapping pathways from the protein targets to nodes in the signaling network by searching a directed protein interaction database and computing a ranked list of pathways by minimizing a cost function based on edge weights along each pathway;
expanding the logic-based differential equation model by incorporating the mapped pathways to create expanded model variants;
simulating each expanded model variant to predict effects of the candidate compounds on the cellular phenotype; and
outputting predictions of how the candidate compounds modulate the cellular phenotype through the identified pathways.

2. The computer-implemented method of claim 1, wherein the cellular phenotype is cardiomyocyte hypertrophy.

3. The computer-implemented method of claim 2, wherein the candidate compounds comprise escitalopram, mifepristone, tirbanibulin, OSI-930, crizotinib, rifabutin, and hesperidin.

4. The computer-implemented method of claim 1, wherein mapping pathways comprises identifying top scoring directed pathways.

5. The computer-implemented method of claim 1, wherein simulating each expanded model variant comprises implementing drug mechanisms of action using logic-based equations that account for drug action as agonist or antagonist and drug binding as competitive or noncompetitive.

6. The computer-implemented method of claim 5, wherein the simulation is run first without drug input to establish baseline activity and then with the drug input to identify steady-state changes in drug response.

7. The computer-implemented method of claim 1, further comprising performing mechanistic subnetwork analysis to identify pathways mediating drug response.

8. The computer-implemented method of claim 7, wherein the mechanistic subnetwork analysis comprises:
simulating a global response of the signaling network to a drug in the presence of a stimulus to identify drug-responsive nodes;
simulating knockdown of each node and measuring that node's influence on drug-induced change in the cellular phenotype to identify regulating nodes; and
determining an intersection of the regulating nodes with the drug-responsive nodes to create a mechanistic subnetwork.

9. The computer-implemented method of claim 1, further comprising validating the expanded model variants against validation data to assess functional validity of the identified drug pathways.

10. The computer-implemented method of claim 1, wherein the candidate compounds are identified from a previous experimental screen for compounds that inhibit the cellular phenotype.

11. The computer-implemented method of claim 1, wherein the predictions identify off-target pathways through which the candidate compounds modulate the cellular phenotype.

12. The computer-implemented method of claim 11, wherein the off-target pathways differ from primary therapeutic mechanisms of the candidate compounds.

13. A drug discovery system comprising:
a processor;
a memory coupled to the processor and storing instructions that, when executed by the processor, cause the processor to:
access a logic-based model of a signaling network for a given condition, the model
including a plurality of nodes and a plurality of edges;
determine a set of candidate compounds that may affect the given condition;
identify protein targets for the candidate compounds using a drug-target database;
determine potential connections from the candidate compounds to the given condition via the logic-based model by computing a ranked list of pathways through searching a directed protein interaction network and minimizing a cost function of edge weights along each pathway;
simulate drug mechanisms of action using logic-based equations accounting for drug action and drug binding properties; and
output the ranked list of pathways and predicted drug effects for drug discovery applications.

14. The drug discovery system of claim 13, wherein simulating drug mechanisms of action comprises implementing logic-based equations that account for drug action as agonist or antagonist and drug binding as competitive or noncompetitive.

15. The drug discovery system of claim 14, wherein the simulation is run first without drug input to establish baseline activity and then with the drug to identify steady-state changes in drug response.

16. The drug discovery system of claim 13, wherein the instructions further cause the processor to perform mechanistic subnetwork analysis to identify pathways mediating drug response.

17. The drug discovery system of claim 16, wherein the mechanistic subnetwork analysis comprises:
simulating a global response of the signaling network to a drug in the presence of a stimulus to identify drug-responsive nodes;
simulating knockdown of each node and measuring that node's influence on drug-induced change in the given condition to identify regulating nodes; and
determining an intersection of the regulating nodes with the drug-responsive nodes to create a mechanistic subnetwork.

18. A method of treating cardiomyocyte hypertrophy in a patient, comprising:
identifying a drug predicted to inhibit cardiomyocyte hypertrophy using a logic-based machine learning method that maps drug targets to a validated cardiomyocyte hypertrophy signaling network model and predicts antihypertrophic pathways through mechanistic subnetwork analysis; and
administering to the patient a therapeutically effective amount of the identified drug, wherein the drug is selected from the group consisting of escitalopram, mifepristone, tirbanibulin, OSI-930, crizotinib, rifabutin, and hesperidin.

19. The method of claim 18, wherein the mechanistic subnetwork analysis comprises:
identifying drug-responsive nodes by simulating global response of the signaling network to the drug in the presence of a hypertrophic stimulus;
identifying regulating nodes by simulating knockdown of each node and measuring influence on drug-induced change in cardiomyocyte hypertrophy; and
determining an intersection of the regulating nodes with the drug-responsive nodes.
